SEQUENCE LISTING (2 sequences)

SEQ ID NO: 1 - DNA, 1326 bp, Homo sapiens
accattaagaggaaagcgatggaggagctgagcgctgacgagattcgacggaggcgcctggcacgacttgctggtggacagacctcccagccgaccaccccgcttacatctccccagagggagaaccctccgggacctccaatagctgcatcagccccaggcccctcccagagtcttggtctcaatgtccacaacatgaccccagctacctcccccataggtgcagcagacaacatcgctgtcagagggttgcatgtaggtcaacaccaccaacttctccccatggactcatccaagaagacagaggtggttctcctggcctgtggctcttttaaccccatcaccaacatgcacctcaggctgttcgagctggccaaggactatatgcatgctacaggaaaatacagtgttatcaaaggcattatctcaccggtcggtgatgcgtacaagaagaaagggctcatcccagcccaccaccgaatcatcatggcagaacttgccaccaagaactcacactgggtggaagtggatacgtgggaaagtcttcagaaggagtgggtggagactgtgaaggtgctcagataccatcaggagaagctggcaactggcagctgcagttacccacaaagctcacctgcactggaaaagcctgggcggaagaggaagtgggctgatcaaaagcaagattctagcccacagaagccccaagagcccaaaccaacaggtgtgcccaaggtgaaattgctgtgtggggcagatttactggagtccttcagcgtgcccaacttgtggaagatggaggacatcacgcaaatcgtggccaactttgggctcatctgtatcactcgggctggcagtgacgctcagaaattcatctacgagtccgatgtgctgtggagacatcagagcaacatccacctggtgaacgagtggatcaccaatgacatctcgtccaccaagatccggagggcgctcaggaggggccagagcatccgctacttggtaccggacctggtccaagagtacattgagaagcatgagctgtacaacacggagagcgaaggcaggaatgctggggtcaccctggctcctctgcagaggaacgccgcagaggccaagcacaaccattccactctgtgacacagggcacggcgtccgcagaggctcgtctggagactcgaaactcagggaaggacttgccatcatcctgtttcatcaactgaaagataaaggttcgatttaaaaaaaaaaacaaaccaccagggaattaagatccgtgactgagatgaatgttttaaataagaccattaaaaaaaaggatgtaat

SEQ ID NO: 2 - protein, 373 aa, Homo sapiens
MEELSADEIRRRRLARLAGGQTSQPTTPLTSPQRENPPGPPIAASAPGPSQSLGLNVHNMTPATSPIGAADNIAVRGLHVGQHHQLLPMDSSKKTEVVLLACGSFNPITNMHLRLFELAKDYMHATGKYSVIKGIISPVGDAYKKKGLIPAHHRIIMAELATKNSHWVEVDTWESLQKEWVETVKVLRYHQEKLATGSCSYPQSSPALEKPGRKRKWADQKQDSSPQKPQEPKPTGVPKVKLLCGADLLESFSVPNLWKMEDITQIVANFGLICITRAGSDAQKFIYESDVLWRHQSNIHLVNEWITNDISSTKIRRALRRGQSIRYLVPDLVQEYIEKHELYNTESEGRNAGVTLAPLQRNAAEAKHNHSTL

(19) United States
(12) Patent Application Publication
(10) Pub. No.: US 2004/0127439 A1
(43) Pub. Date: Jul. 1, 2004

(54) Introduction of the wld gene for prevention of axonal degeneration in neurological diseases

(76) Inventors: Jonathan D Glass, Atlanta, GA (US); Mark M Rich, Decatur, GA (US)

Correspondence Address: Christopher B Linder, Thomas Kayden Horstmeyer & Risley, Suite 1750, 100 Galleria Parkway NW, Atlanta, GA, 30339-5948, US

(21) Appl. No.: 10/433,035

(22) PCT Filed: Oct. 1, 2002
(86) PCT No.: PCT/US02/31078

(51) Int. Cl. A61K 48/00

(52) U.S. Cl. 514/44

(57) ABSTRACT

Polypeptides that can protect axons from axon degeneration and methods of use of the polypeptides are presented. Polynucleotides that can protect axons from axon degeneration and methods of use of the polynucleotides are presented. In addition, polynucleotides that encode the polypeptides referred to above are presented. Further, pharmaceutical compositions to treat conditions are presented.

### CROSS-REFERENCE TO RELATED APPLICATION

This application claims priority to copending U.S. provisional application entitled, “INTRODUCTION OF THE WLD GENE FOR PREVENTION OF AXONAL DEGNERATION IN NEUROLOGICAL DISEASES,” having ser. No. 60/326,354, filed Oct. 1, 2001, which is entirely incorporated herein by reference.

### STATEMENT REGARDING FEDERALLY SPONSORED RESEARCH OR DEVELOPMENT

The U.S. government has a paid-up license in this invention and the right in limited circumstances to require the patent owner to license others on reasonable terms as provided for by the terms of NS-38848 awarded by the National Institutes of Health (NIH) of the U.S.

### TECHNICAL FIELD

The present invention is generally related to polynucleotides and polypepetides and, more particularly, is related to polynucleotides and polypepetides relating to the prevention and/or treatment of axonal degeneration.

### BACKGROUND

Axonal degeneration is a pathological substrate leading to loss of neurological function in a wide variety of acute and chronic disorders of the central nervous system (CNS) and peripheral nervous system (PNS). Diseases as disparate as stroke, spinocerebellar degenerations, and peripheral neuropathies share the common pathological finding of axonal degeneration. Even in primary demyelinating disorders such as multiple sclerosis and hereditary motor sensor neuropathies (e.g., HMSN-1), axonal degeneration is the pathological finding most highly correlated with severity of clinical symptoms. The mechanisms underlying axonal degeneration in all of these exemplary disorders are unknown.

Wallerian degeneration is the simplest and most thoroughly studied model of axonal degeneration. Previous studies have demonstrated that degradation of the axonal cytoskeleton in axotomized nerve fibers is a calcium-dependent process. In experimental systems, reduction of calcium to below a critical threshold of 200 μM delays the onset of axotomy-induced axonal degeneration. Calcium entry likely activates axonal calpains. “Calpains” are ubiquitous calcium-dependent cysteine proteases involved in both physiological and pathological cellular functions. In experimental Wallerian degeneration, administration of calpain inhibitors is protective against axonal degeneration.

The pathological features of the majority of human peripheral neuropathies are similar to those seen in axotomy-induced Wallerian degeneration. This similarity has led investigators to describe many neuropathies as “Wallerian-like” degeneration. It has been hypothesized that similar mechanisms are involved in axonal degeneration seen in peripheral neuropathies and in Wallerian degeneration, and that strategies for protecting against axotomy-induced axonal degeneration may be protective in peripheral neuropathies. In order to investigate the roles of calcium and calpains in peripheral neuropathy, an in vitro model of toxin-induced axonal degeneration using the neurotoxin vincristine and been developed and tested to determine the neuroprotective effects of a low calcium environment and calpain inhibition.

Vincristine is a chemotherapeutic agent used to treat leukemias and other types of human cancers. Patients treated with vincristine predictably develop neuropathic symptoms and signs, the most prominent of which are distal-extremity paresthesias, sensory loss, and reduction of deep tendon reflexes. Pathologically, vincristine causes length-dependent axonal degeneration that is typical of many other drug-induced, metabolic, and idiopathic peripheral neuropathies.

The slow Wallerian degeneration (Wld^{S}) mouse is a spontaneously occurring mutant strain of mouse that demonstrates the remarkable phenotype of prolonged axonal survival following nerve injury in the central nervous system (CNS) and peripheral nervous system (PNS). The Wld^{S} mutation is created by the splicing of fragments of two genes, Ufd2 and D4 Colele, within an 85 kb triplication on cluomosome 4. This splice creates a new open reading frame and codes for a 42 kD chimeric protein that is unique to the Wld^{S} mouse. The specific function of this protein was unknown.

Thus, a heretofore unaddressed need exists in the industry to address the aforementioned deficiencies and inadequacies.

### SUMMARY OF THE INVENTION

Briefly described, embodiments of the present invention include polynucleotides and polypeptides that can be used to treat axonal degeneration and related diseases. In addition, the present invention provides for probes, expression vectors, antibodies, and fusion proteins that are described in more detail below.

A representative embodiment of the present invention includes a method of preventing axonal degeneration in a host having a nervous system dysfunction. The method includes administering to the host a therapeutically effective amount of a composition that includes a polynucleotide selected from: a polynucleotide sequence set forth in SEQ ID NO:1, or a degenerate variant of the SEQ ID NO:1; a polynucleotide sequence at least 90% identical to the polynucleotide sequence set forth in SEQ ID NO:1; a polynucleotide sequence at least 75% identical to the polynucleotide sequence set forth in SEQ ID NO:1; and a polynucleotide sequence at least 50% identical to the polynucleotide sequence set forth in SEQ ID NO:1.

In another embodiment, the present invention provides for a method of preventing axonal degeneration in a host having a nervous system dysfunction that inlcudes administering to the host a therapeutically effective amount of a composition. The composition includes a polypeptide selected from: an amino acid sequence set forth in SEQ ID NO:2, or conservatively modified variants thereof; an amino acid sequence that is at least 90% identical to SEQ ID NO:2; an amino acid sequence that is at least 75% identical to SEQ ID NO:2; and an amino acid sequence that is at least 50% identical to SEQ ID NO:2.

In still another embodiment, the present invention provides for a method of protecting axons from axon degeneration by exposing the axons to a composition. The composition includes a polynucleotide selected from: a polynucleotide sequence set forth in SEQ ID NO:1, or a degenerate variant of the SEQ ID NO:1; a polynucleotide sequence at least 90% identical to the polynucleotide sequence set forth in SEQ ID NO:1; a polynucleotide sequence at least 75% identical to the polynucleotide sequence set forth in SEQ ID NO:1; and a polynucleotide sequence at least 50% identical to the polynucleotide sequence set forth in SEQ ID NO: 1.

In still another embodiment, the present invention provides for a method of protecting axons from axon degeneration by exposing the axons to a composition. The composition includes a polypeptide selected from: an amino acid sequence set forth in SEQ ID NO:2, or conservatively modified variants thereof; an amino acid sequence that is at least 90% identical to SEQ ID NO:2; an amino acid sequence that is at least 75% identical to SEQ ID NO:2; and an amino acid sequence that is at least 50% identical to SEQ ID NO:2.

In still another embodiment, the present invention provides for a method of treating a condition comprising administering to a host in need of treatment an effective amount of a polypeptide. The polypeptide is selected from: an amino acid sequence set forth in SEQ ID NO:2, or conservatively modified variants thereof; an amino acid sequence that is at least 90% identical to SEQ ID NO:2; an amino acid sequence that is at least 75% identical to SEQ ID NO:2; and an amino acid sequence that is at least 50% identical to SEQ ID NO:2.

In still another embodiment, the present invention provides for a method of treating a condition comprising administering to a host in need of treatment an effective amount of a polynucleotide. The polynucleotide is selected from: a polynucleotide sequence set forth in SEQ ID NO:1, or a degenerate variant of the SEQ ID NO:1; a polynucleotide sequence at least 90% identical to the polynucleotide sequence set forth in SEQ ID NO:1; a polynucleotide sequence at least 75% identical to the polynucleotide sequence set forth in SEQ ID NO:1; and a polynucleotide sequence at least 50% identical to the polynucleotide sequence set forth in SEQ ID NO:1.

In still another embodiment, the present invention provides for a pharmaceutical composition comprising a polypeptide in combination with a pharmaceutically acceptable carrier. The polypeptide is selected from: an amino acid sequence set forth in SEQ ID NO:2, or conservatively modified variants thereof; an amino acid sequence that is at least 90% identical to SEQ ID NO:2; an amino acid sequence that is at least 75% identical to SEQ ID NO:2; and an amino acid sequence that is at least 50% identical to SEQ ID NO:2.

In still another embodiment, the present invention provides for a pharmaceutical composition comprising a polynucleotide in combination with a pharmaceutically acceptable carrier. The polynucleotide is selected from: a polynucleotide sequence set forth in SEQ ID NO:1, or a degenerate variant of the SEQ ID NO:1; a polynucleotide sequence at least 90% identical to the polynucleotide sequence set forth in SEQ ID NO:1; a polynucleotide sequence at least 75% identical to the polynucleotide sequence set forth in SEQ ID NO:1; and a polynucleotide sequence at least 50% identical to the polynucleotide sequence set forth in SEQ ID NO:1.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention.

FIG. 1 is a serial montage phase-contrast photomicrographs of the same DRG exposed to 0.01 μM vincristine. There is a progressive “dying back” of neurites from day 0 to day 6.

FIG. 2 is a graphic representation of changes in DRG area (mm²) and neurite length (mm) with vincristine exposure alone, or with addition of 2 mM EGTA or 50 μM AK295. Growth arrest can be noted even with addition of neuroprotective drugs.

FIG. 3 is a direct comparison of morphology on day 3 of cultures exposed to vincristine alone (A), or with addition of AK295 (B) or EGTA (C). Axonal degeneration is apparent even in the treated cultures, but is noticeably less than in the untreated culture. DRGs are stained with MAP-S antibody.

FIG. 4 is a graph of a quantitative measure of axonal survival with EGTA and AK295 in axotomized neurites. Unaxotomized control cultures are arbitrarily given a score of 4 to separate them from the experimental axotomized cultures. Numbers in parentheses are the number of experiments performed.

FIGS. 5A-F is a comparison of protective effects of AK295 in vincristine neuropathy (5A-C) and Wallerian degeneration (5D-F). FIGS. 5A and 5D are umnanipulated controls (11 days in culture), 5 and 5E are untreated cultures exposed to vincristine for 6 days (5) or axotomized for 3 days (5E). FIGS. 5C and 5F are the same respective injuries in media containing 50 μM AK 295. The scale bars in FIG. 5C are the same for all images.

FIG. 6 is representative photo-montages of DRG cultures comparing CS7BL/6 to Wld^{S} after exposure to vincristine (0.05 μM) for 4 days. Measurements were made using these types of images for area of DRG halo (circle) and length of longest axon (arrow).

FIG. 7 is dose-dependent response of cultured Wld^{S} and C57BL/6 axons to vincristine. Comparisons of individual groups are shown on the graph: *p<0.01, compared to 0.01 μM vincristine; # p<0.01, compare to 0.02 μM vincristine. Data are mean±SEM.

FIG. 8 are comparisons of normalized data for halo area and axon length at each dose of vincristine. Data are graphed as mean±SEM. *p<0.05; # p<0.01; $ p<0.001; ‡ p<0.0001; NS, not significant.

FIGS. 9A and 9B are comparisons of sensory neurite growth (length and area) in C57BL/6 (solid lines) and Wld^{S} axons (dotted lines). For FIG. 9A, there were no differences in growth characteristics of unexposed cultures. For FIG. 9B, cultures exposed to 0.05 μM vincristine for 24 hours demonstrated significant differences between C57BL/6 and Wld^{S} throughout the 20 day observation period (p<0.001). C57BL/6 neurites showed no recovery. In Wld^{S} cultures significant growth could be demonstrated (*p<0.01) at days 12, 16, and 20 when compared to day 4. Y axis: percent of day 0, +/−SEM. X-axis: days after exposure to vincristine.

FIGS. 10A, A′, B and B′ are representative photomicrographs demonstrating axonal growth in a vincristine-exposed Wld^{S} culture. The same DRG is shown at day 10 (FIGS. 10A and A′) and day 20 (Figs. B and B′). The arrows depict the same point on an individual axon at days 10 and 20, and the arrowheads point to the axon terminal at these two stages. Note the extension of this neurite. The apparent reduction in density of neurites at day 20 as compared to day 10 is likely due to “spreading out” of growing neurites.

FIGS. 11A, B, and C are Western blots using the polyclonal Wld^{S} antibody demonstrating adenoviral expression of the Wld^{S} protein in HEK 293A cells (FIG. 11A) and rat DRG neurons (FIG. 11B). Several non-specific bands appear in the blot from neuronal tissue. Immunofluorescence shows the expression of the Wld^{S} protein only in cultures exposed to the adenovirus containing the Wld^{S} gene (FIG. 11C). The overlay panel demonstrates that the protein expression is in axons.

FIG. 12 depicts the protective effect of expression of Wld^{S} in rat DRG cultures. Uninfected DRG (solid line) and cultures infected with the control adenovirus expressing only lacZ (dotted line) rapidly die when exposed to 0.01 μM vincristine. Cultures infected with the Wld^{S} expressing adenovirus (dashed line) show significant resistance to vincristine toxicity at all time points tested.

FIGS. 13A, B and C are photomicrographs of representative cultures, demonstrating the pathological effects of vincristine exposure after 10 days in uninfected cultures (FIG. 13A), cultures infected with control adenovirus (FIG. 13B), and cultures infected with the Wld^{S} expressing adenovirus (FIG. 13C). The arrows point to the extent of axonal growth. The inset in FIG. 13C is a higher power view demonstrating the continuity of axons. Cultures are stained with MAP-5 for identification of neurites.

### DETAILED DESCRIPTION

Embodiments of the present invention provide for polypeptides and polynucleotides that can be used to prevent and/or treat a number of neurological diseases, disorders, and symptoms.

Prior to setting forth embodiments of the invention in detail, it may be helpful to first define the following terms:

The term “affinity tag” is used herein to denote a polypeptide segment that can be attached to a second polypeptide to provide for purification of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson, et al., EMBO J. 4:1075, 1985; Nilsson, et al., Methods Enzymol., 198:3, 1991), glutathione S transferase (Smith, et al., Gene, 67:31, 1988), Glu-Glu affinity tag, substance P, Flag™ peptide (Hopp, et al., Biotechnology, 6:1204-10, 1988), streptavidin binding peptide, or other antigenic epitope or binding domain. See, in general, Ford, et al., Protein Expression and Purification, 2: 95-107, 1991. DNAs encoding affinity tags are available from commercial suppliers (e.g., Pharmacia Biotech, Piscataway, N.J.).

“Polynucleotide” generally refers to any polyribonucleotide or polydeoxribonucleotide, which may be unmodified ribonucleic acid (RNA) or deoxyribonucleic acid (DNA) or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or INAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically, or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, (i.e., peptide isosteres). “Polypeptide” refers to both short chains, commonly referred to as peptides, oligopeptides, or oligomers, and to longer chains, generally referred to as proteins. “Polypeptides” may contain amino acids other than the 20 gene-encoded amino acids. “Polypeptides” include amino acid sequences modified either by natural processes, such as post-translational processing, or by chemical modification techniques, which are well known in the art. Such modifications are described in basic texts and in more detailed monographs, as well as in a voluminous research literature.

Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched, and branched cyclic polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (Proteins—Structure and Molecular Properties, 2nd Ed., T. E. Creighton, W.H. Freeman and Company, New York, 1993; Wold, F., Post-translational Protein Modifications: Perspectives and Prospects, pgs. 1-12 in Post-translational Covalent Modification of Proteins, B. C. Johnson, Ed., Academic Press, New York, 1983; Seifter, et al., Meth Enzymol, 182: 626-646, 1990, and Rattan, et al., Ann NY Acad. Sci., 663:48-62, 1992).

“Variant” refers to a polynucleotide or polypeptide that differs from a reference polynucleotide or polypeptide, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions, and truncations in the polypeptide encoded by the reference sequence, as discussed below.

A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, and deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data. Part I, Griffin, A. M., and Griffin, H. G., Eds., Humana Press, New Jersey, 1994; Sequence Analysis in Molecular Biology,von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polynucleotides and polypeptides of the present invention.

By way of example, a polynucleotide sequence of the present invention may be identical to the reference sequence of SEQ ID NO:1, that is be 100% identical, or it may include up to a certain integer number of nucleotide alterations as compared to the reference sequence. Such alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in the reference nucleotide by the numerical percent of the respective percent identity (divided by 100) and subtracting that product from said total number of nucleotides in the reference nucleotide. Alterations of a polynucleotide sequence encoding the polypeptide may alter the polypeptide encoded by the polynucleotide following such alterations.

Similarly, a polypeptide sequence of the present invention may be identical to the reference sequence of SEQ ID NO:2, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (e.g., GAU and GAC triplets each encode Asp).

The term “expression vector” is used to denote a DNA molecule, linear or circular, which includes a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated polynucleotide molecules of the present invention are free of other polynucleotides with which they are ordinarily associated, but may include naturally occurring 5′ and 3′ untranslated regions such as promoters and terminators. The identification of associated regions will be evident to one of ordinary skill in the art (Dynan, et al., Nature, 316: 774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

The term “operably linked”, when referring to DNA segments, indicates that the segments are arranged so that they function in concert for their intended purposes (e.g., transcription initiates in the promoter and proceeds through the coding segment to the terminator).

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The term “condition” and “conditions” denote a state of health that can be related to processes involving the axonal degeneration and nervous system disorder. The processes that involve axonal degeneration and nervous system disorder are discussed below, and are to be included as condition(s) that can be treated by embodiments of the present invention.

The term “host” includes both humans, mammals (e.g., cats, dogs, horses, etc.), and other living species that are in need of treatment. Hosts that are “predisposed to” condition(s) can be defined as hosts that do not exhibit overt symptoms of one or more of these conditions but that are genetically, physiologically, or otherwise at risk of developing one or more of these conditions.

The term “treat”, “treating”, and “treatment” are an approach for obtaining beneficial or desired clinical results. For purposes of embodiments of this invention, beneficial or desired clinical results include, but are not limited to, alleviation of symptoms, diminishment of extent of disease, stabilization (i.e., not worsening) of disease, preventing spread (i.e., metastasis) of disease, delaying or slowing of disease progression, amelioration or palliation of the disease state, and remission (partial or total) whether detectable or undetectable. In addition, “treat”, “treating”, and “treatment” can also mean prolonging survival as compared to expected survival if not receiving treatment.

The term “modulate” and “modulation” denote adjustment or regulation of the activity of a compound or the interaction between one or more compounds.

The term “phenotype” means a property of an organism that can be detected, which is usually produced by interaction of an organism's genotype and environment.

The term “open reading frame” means the amino acid sequence encoded between translation initiation and termination codons of a coding sequence.

The term “codon” means a specific triplet of mononucleotides in the DNA chain. Codons correspond to specific amino acids or to start and stop of translation by the ribosome.

The term “wild-type” means that the nucleic acid fragment does not comprise any mutations. A “wild-type” protein means that the protein will be active at a level of activity found in nature and will comprise the amino acid sequence found in nature.

The term “chimeric protein” means that the protein comprises regions which are wild-type and regions which are mutated. It may also mean that the protein comprises wild-type regions from one protein and wild-type regions from another related protein.

The term “mutation” means a change in the sequence of a wild-type nucleic acid sequence or a change in the sequence of a peptide. Such mutation may be a point mutation such as a transition or a transversion. The mutation may be a deletion, an insertion or a duplication.

In the polypeptide notation used herein, the lefthand direction is the amino terminal direction and the righthand direction is the carboxy-terminal direction, in accordance with standard usage and convention. Similarly, unless specified otherwise, the lefthand end of single-stranded polynucleotide sequences is the 5′ end; the lefthand direction of double-stranded polynucleotide sequences is referred to as the 5′ direction.

The term “agent” is used herein to denote a chemical compound, a mixture of chemical compounds, an array of spatially localized compounds (e.g., a VLSIPS peptide array, polynucleotide array, and/or combinatorial small molecule array), a biological macromolecule, a bacteriophage peptide display library, a bacteriophage antibody (e.g., scFv) display library, a polysome peptide display library, or an extract made from biological materials such as bacteria, plants, fungi, or animal (particularly mammalian) cells or tissues.

All publications, including but not limited to patents and patent applications, cited in this specification are herein incorporated by reference as if each individual publication were specifically and individually indicated to be incorporated by reference herein as though fully set forth.

Many neurological diseases are characterized by axonal degeneration. As noted above, Wallerian degeneration is model of axonal degeneration. Embodiments of the present invention is generally directed using polypeptides and polynucleotides (e.g., Wallerian degeneration (Wld^{S}) gene and the corresponding protein) to slow or cease Wallerian degeneration. In the process of the present invention, the polypeptides and polynucleotides is inserted into a vector or otherwise introduced to the host, which is used to infect an host's tissue so that the gene expresses the Wld^{S} protein. Thus, when the host is subjected to disease or agent that would normally cause nerve injury in the central nervous system (CNS) and peripheral nervous system (PNS), the Wallerian degeneration is slowed and, in some cases, even stopped. The process of the present invention results in a degree of axonal degeneration that is significantly less than that occurring in organisms without the Wld^{S} gene.

In general, the polypeptides and polynucleotide of the present invention can be used to treat disorders, such as, but not limited to, degenerative, heritable, and metabolic disorders. Particular examples of disorders that can be treated with the process and polypeptides and polynucleotide of the present invention include but are not limited to, peripheral and specific neuropathies, direct axonal injury, trauma and ischemia, stroke, Alzheimer's disease, Charcot-Marie-Tooth, chronic spinocerebellar degeneration and primary demyelinating diseases, such as for example multiple sclerosis.

Polypeptides and Polynucleotides

As indicated above, embodiments of the present invention include polypeptides and polynucleotides that encode the polypeptides. Embodiments of the polypeptide are designated “WLDS polypeptides”, while embodiments of the polynucleotides are designated “WLDS polynucleotides.” The WLDS polynucleotide sequence is set forth in SEQ ID NO:1 and the corresponding WLDS polypepetide amino acid sequence is set forth in SEQ ID NO:2.

As discussed above, embodiments of the present invention provide WLDS polynucleotides, including DNA and RNA molecules that encode the WLDS polypeptides. Those skilled in the art will readily recognize that, in view of the degeneracy of the genetic code, considerable sequence variation is possible among these polynucleotide molecules. SEQ ID NO:1 is a degenerate polynucleotide sequence that encompasses polynucleotide that encodes the WLDS polypeptide of SEQ ID NO:2. The degeneracy of nucleic acid is well known in the art and as such degenerate polynucleotides of SEQ ID NO:1 are included within the scope of the present invention.

Table 1 sets for the three letter symbols and the one letter symbols for the amino acids as well as possible codons that can be associated with the amino acids.

TABLE 1

| THREE | ONE LETTER | SYNONYMOUS |
|---|---|---|
| LETTER CODE | CODE | CODONS |
| Cys | C | TGC TGT |
| Ser | S | AGC AGT TCA TCC |
| | | TCG TCT |
| Thr | T | ACA ACC ACG ACT |
| Pro | P | CCA CCC CCG CCT |
| Ala | A | GCA GCC GCG GCT |
| Gly | G | GGA GGC GGG GGT |
| Asn | N | AAC AAT |
| Asp | D | GAC GAT |
| Glu | E | GAA GAG |
| Gln | Q | CAA CAG |
| His | H | CAC CAT |
| Arg | R | AGA AGG CGA CGC |
| | | CGG CGT |
| Lys | K | AAA AAG |
| Met | M | ATG |
| Ile | I | ATA ATC ATT |
| Leu | L | CTA CTC CTG CTT |
| | | TTA TTG |
| Val | V | GTA GTC GTG GTT |
| Phe | F | TTC TTT |
| Tyr | Y | TAC TAT |
| Trp | W | TGG |
| Asn-Asp | B | |
| Glu-Gln | Z | |
| Any | X | |

One of ordinary skill in the art will appreciate that some ambiguity is introduced in determining a degenerate codon. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NO:2.

Variant WLDS polynucleotides that encode polypeptides that can treat a condition as defined above are within the scope of the embodiments of the present invention. More specifically, variant WLDS polynucleotides that encode polypeptides which exhibit at least about 50%, about 75%, about 85%, and preferably about 90%, of the activity of WLDS polypeptides encoded by the variant WLDS polynucleotide are within the scope of the embodiments of the present invention.

For any WLDS polypeptide, including variants and fusion proteins, one of ordinary skill in the art can readily generate a fully degenerate polynucleotide sequence encoding that variant using the information set forth in Table 1. Moreover, those of skill in the art can use standard software to devise WLDS variants (i.e., polynucleotides and polypeptides) based upon the polynucleotide and amino acid sequences described herein.

As indicated above, WLDS polynucleotides and isolated WLDS polynucleotides of the present invention can include DNA and RNA molecules. Methods for preparing DNA and RNA are well known in the art. In general, RNA is isolated from a tissue or cell that produces WLDS RNA. Such tissues and cells can be identified by Northern blotting (Thomas, Proc. Natl. Acad. Sci. USA, 77: 5201, 1980). An exemplary source being human testis tissue. Total RNA can be prepared using guanidine HCl extraction followed by isolation by centrifugation in a CsCl gradient (Chirgwin, et al., Biochemistry, 18:52-94, 1979). Complementary DNA (cDNA) can be prepared from the RNA using known methods. In the alternative, genomic DNA can be isolated. Polynucleotides encoding WLDS polypeptides are then identified and isolated by hybridization or PCR, for example.

WLDS polynucleotides can also be synthesized using techniques widely known in the art. (Glick, et al., Molecular Biotechnology Principles & Applications of Recombinant DNA, (ASM Press, Washington, D.C. 1994); Itakura, et al., Annu. Rev. Biochem., 53: 323-56, 1984 and Climie, et al., Proc. Natl. Acad. Sci. USA, 87: 633-7, 1990.

Embodiments of the present invention also provide for WLDS polypeptides and isolated WLDS polypeptides that are substantially homologous to the WLDS polypeptide of SEQ ID NO:2. The term “substantially homologous” is used herein to denote polypeptides having about 50%, about 75%, about 85%, and preferably about 90% sequence identity to the sequence shown in SEQ ID NO:2. Percent sequence identity is determined by conventional methods as discussed above. In addition, embodiments of the present invention include polynucleotides that encode homologous polypeptides.

In general, homologous polypeptides are characterized as having one or more amino acid substitutions, deletions, and/or additions. These changes are preferably of a minor nature, that is conservative amino acid substitutions and other substitutions that do not significantly affect the activity of the polypeptide; small substitutions, typically of one to about six amino acids; and small amino- or carboxyl-terminal extensions, such as an amino-terminal methionine residue, a small linker peptide of up to about 2-6 residues, or an affinity tag. Homologous polypeptides comprising affinity tags can further comprise a proteolytic cleavage site between the homologous polypeptide and the affinity tag.

In addition, embodiments of the present invention include polynucleotides that encode polypeptides having one or more “conservative amino acid substitutions,” compared with the WLDS polypeptide of SEQ ID NO:2. Conservative amino acid substitutions can be based upon the chemical properties of the amino acids. That is, variants can be obtained that contain one or more amino acid substitutions of SEQ ID NO:2., in which an alkyl amino acid is substituted for an alkyl amino acid in a WLDS polypeptide, an aromatic amino acid is substituted for an aromatic amino acid in a WLDS polypeptide, a sulfur-containing amino acid is substituted for a sulfur-containing amino acid in a WLDS polypeptide, a hydroxy-containing amino acid is substituted for a hydroxy-containing amino acid in a WLDS polypeptide, an acidic amino acid is substituted for an acidic amino acid in a WLDS polypeptide, a basic amino acid is substituted for a basic amino acid in a WLDS polypeptide, or a dibasic monocarboxylic amino acid is substituted for a dibasic monocarboxylic amino acid in a WLDS polypeptide.

Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. Other conservative amino acid substitutions are provided in Table 2.

TABLE 2

| CHARACTETISTIC | AMINO ACID |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

Conservative amino acid changes in WLDS polypeptides can be introduced by substituting nucleotides for the nucleotides recited in SEQ ID NO:1. Such “conservative amino acid” variants can be obtained, for example, by oligonucleotide-directed mutagenesis, linker-scanning mutagenesis, mutagenesis using the polymerase chain reaction, and the like (McPherson (Ed.), Directed Mutagenesis: A Practical Approach (IRL Press 1991)). The ability of such variants to treat conditions as well as other properties of the wild-type protein can be determined using a standard methods. Alternatively, variant WLDS polypeptides can be identified by the ability to specifically bind anti-WLDS antibodies.

WLDS polypeptides having conservative amino acid variants can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methyl-glycine, allo-threonine, methylthreonine, hydroxy-ethylcysteine, hydroxyethylhomocysteine, nitro-glutamine, homoglutamine, pipecolic acid, thiazolidine carboxylic acid, dehydroproline, 3- and 4-methylproline, 3,3-dimethylproline, tert-leucine, norvaline, 2-azaphenyl-alanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. (Robertson, et al., J. Am. Chem. Soc., 113: 2722, 1991; Ellman, et al., Methods Enzymol., 202: 301, 1991; Chung, et al., Science, 259: 806-9, 1993; and Chung, et al., Proc. Natl. Acad. Sci. USA, 90: 10145-9, 1993). In a second method, translation is carried out in Xenopus oocytes by microinjection of mutated mRNA and chemically aminoacylated suppressor tRNAs (Turcatti, et al., J. Biol. Chem., 271: 19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. (Koide, et al., Biochem., 33: 7470-6, 1994). Naturally occurring amino acid residues can be converted to non-naturally occurring species by in vitro chemical modification. Chemical modification can be combined with site-directed mutagenesis to further expand the range of substitutions (Wynn, et al., Protein Sci., 2: 395-403, 1993).

A limited number (i.e., less than 6) of non-conservative amino acids, amino acids that are not encoded by the genetic code, non-naturally occurring amino acids, and unnatural amino acids may be substituted for WLDS polypeptide amino acid residues.

Essential amino acids in the polypeptides of the present invention can be identified according to procedures known in the art, such as site-directed mutagenesis or alanine-scanning mutagenesis (Cunningham, et al., Science, 244: 1081-5, 1989; Bass, et al., Proc. Natl. Acad. Sci. USA, 88: 4498-502, 1991). In the latter technique, single alanine mutations are introduced at every residue in the molecule, and the resultant mutant molecules are tested for biological activity as disclosed below to identify amino acid residues that are critical to the activity of the molecule. (Hilton, et al., J. Biol. Chem., 271: 4699-708, 1996). Sites of ligand-receptor interaction can also be determined by physical analysis of structure, as determined by such techniques as nuclear magnetic resonance, crystallography, electron diffraction or photoaffinity labeling, in conjunction with mutation of putative contact site amino acids. (de Vos, et al., Science, 255: 306-12, 1992; Smith, et al., J. Mol. Biol., 224: 899-904, 1992; Wlodaver, et al., FEBS Lett., 309: 59-64, 1992). The identities of essential amino acids can also be inferred from analysis of homologies with related nuclear membrane bound proteins.

Multiple amino acid substitutions can be made and tested using known methods of mutagenesis and screening, such as those disclosed by Reidhaar-Olson and Sauer (Science, 241: 53-7, 1988) or Bowie and Sauer (Proc. Natl. Acad. Sci. USA, 86: 2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (Lowman, et al., Biochem., 30: 10832-7, 1991; Ladner, et al., U.S. Pat. No. 5,223,409) and region-directed mutagenesis (Derbyshire, et al., Gene, 46:145, 1986; Ner, et al., DNA, 7:127, 1988).

Variants of the disclosed WLDS polypeptide can be generated through DNA shuffling. (Stemmer, Nature, 370: 389-91, 1994 and Stemmer, Proc. Natl. Acad. Sci. USA, 91: 10747-51, 1994). Briefly, variant polypeptides are generated by in vitro homologous recombination by random fragmentation of a parent DNA followed by reassembly using PCR, resulting in randomly introduced point mutations. This technique can be modified by using a family of parent DNAs, such as allelic variants or genes from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods can be combined with high-throughput, automated screening methods to detect activity of cloned, mutagenized polypeptides in host cells. Preferred assays in this regard include cell proliferation assays and biosensor-based ligand-binding assays. Mutagenized DNA molecules that encode active polypeptides can be recovered from the host cells and rapidly sequenced using modern equipment. These methods allow the rapid determination of the importance of individual amino acid residues in a polypeptide of interest, and can be applied to polypeptides of unknown structure.

Using the methods discussed herein, one of ordinary skill in the art can identify and/or prepare a variety of WLDS polypeptide fragments or variants of SEQ ID NO:2 that retain the functional properties of the WLDS polypeptide. Such polypeptides may also include additional polypeptide segments as generally disclosed herein.

For any WLDS polypeptide, including variants and fusion proteins, one of ordinary skill in the art can readily generate a degenerate polynucleotide sequence encoding that variant using the information set forth in Table 1 above as well as what is known in the art.

As used herein, a fusion protein consists essentially of a first portion and a second portion joined by a peptide bond. In one embodiment the first portion includes a polypeptide comprising a sequence of amino acid residues that is at least about 50%, about 75%, about 85%, and preferably about 90% identical in amino acid sequence to SEQ ID NO:2 and the second portion is any other heterologous non WLDS polypeptide. The other polypeptide may be polypeptides that do not inhibit the function of the WLDS polypeptide, such as a signal peptide to facilitate secretion of the fusion protein or an affinity tag.

The WILDS polypeptides of the present invention, including full-length polypeptides, biologically active fragments, and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells. (Sambrook et al., Molecular Cloning: A Laboratory Manual, 2nd Ed., Cold Spring Harbor Laboratory Press, Cold Spring Harbor, N.Y., 1989, and Ausubel, et al., Eds., Current Protocols in Molecular Biology, John Wiley and Sons, Inc., N.Y., 1987).

In general, WLDS polynucleotides sequence encoding WLDS polypeptides are operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a WLDS polypeptide into the secretory pathway of a host cell, a secretory signal sequence (also known as a leader sequence, signal sequence, prepro sequence or pre sequence) is provided in the expression vector. The secretory signal sequence may be derived from another secreted protein or synthesized de novo. The secretory signal sequence is operably linked to the WLDS polynucleotide sequence, (i.e., the two sequences are joined in the correct reading frame and positioned to direct the newly synthesized polypeptide into the secretory pathway of the host cell). Secretory signal sequences are commonly positioned 5′ to the polynucleotide sequence encoding the polypeptide of interest, although certain secretory signal sequences may be positioned elsewhere in the polynucleotide sequence of interest (U.S. Pat. No. 5,037,743, U.S. Pat. No. 5,143,830).

It is preferred to purify the WLDS polypeptides of the present invention to about 80% purity, more preferably to about 90% purity, even more preferably about 95% purity, and particularly preferred is a pharmaceutically pure state, that is greater than 99.9% pure with respect to contaminating macromolecules, particularly other proteins and nucleic acids, and free of infectious and pyrogenic agents. Preferably, a purified polypeptide is substantially free of other polypeptides, particularly other polypeptides of animal origin.

Expressed recombinant WLDS polypeptides (or fusion WLDS polypeptides) can be purified using fractionation and/or conventional purification methods and media. Ammonium sulfate precipitation and acid or chaotrope extraction may be used for fractionation of samples. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Methods for binding receptor polypeptides to support media are well known in the art. Selection of a particular method is a matter of routine design and is determined in part by the properties of the chosen support. (Affinity Chromatography: Principles & Methods, Pharmacia LKB Biotechnology, Uppsala, Sweden, 1988).

The WLDS polypeptides of the present invention can be isolated by exploitation of their binding properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem., 3: 1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., 182, M. Deutscher, (Ed.), Acad. Press, San Diego, 1990, pp.529-39). Within additional embodiments of the invention, a fusion of the polypeptide of interest and an affinity tag (e.g., Glu-Glu tag) may be constructed to facilitate purification.

WLDS polypeptides or fragments thereof may also be prepared through chemical synthesis according to methods known in the art, including exclusive solid phase synthesis, partial solid phase methods, fragment condensation or classical solution synthesis. (Merrifield, J. Am. Chem. Soc., 85: 2149, 1963).

Using methods known in the art, WLDS polypeptides may be prepared as monomers or multimers; glycosylated or non-glycosylated; and pegylated or non-pegylated.

An in vivo approach for assaying WLDS polypeptides involves viral delivery systems. Exemplary viruses for this purpose include adenovirus, herpesvirus, vaccinia virus, and adeno-associated virus (AAV). Adenovirus, a double-stranded DNA virus, is currently the best studied gene transfer vector for delivery of heterologous nucleic acid (Becker, et al., Meth. Cell Biol., 43: 161-89, 1994; and Douglas, et al., Science & Medicine, 4: 44-53). The adenovirus system offers several advantages: adenovirus can (i) accommodate relatively large DNA inserts; (ii) be grown to high-titer; (iii) infect a broad range of mammalian cell types; and (iv) be used with a large number of available vectors containing different promoters. Also, because adenoviruses are stable in the bloodstream, they can be administered by intravenous injection.

The WLDS polypeptide can be inserted into portions of the adenovirus by deleting a portion of the adenovirus genome. The WLDS polypeptide may be incorporated into the viral DNA by direct ligation or by homologous recombination with a co-transfected plasmid. In an exemplary system, the essential E1 gene has been deleted from the viral vector, and the virus will not replicate unless the E1 gene is provided by the host cell (the human 293 cell line is exemplary). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a secretory signal sequence is present, secrete) the WLDS polypeptide or conjugates of the WLDS polypeptide. Secreted proteins will enter the circulation in the highly vascularized liver, and effects on the condition to be treated can be determined.

WLDS polypeptides can also be used to prepare antibodies that may inhibit axonal degeneration. The WLDS polypeptide or a fragment thereof serves as an antigen (immunogen) to inoculate an animal and elicit an immune response. Suitable antigens would be the WLDS polypeptide encoded by SEQ ID NO:2, for example. Antibodies generated from this immune response can be isolated and purified as described herein. Methods for preparing and isolating polyclonal and monoclonal antibodies are well known in the art. (Current Protocols in Immunology, Cooligan, et al. (Eds.), National Institutes of Health, John Wiley and Sons, Inc., 1995; Sambrook, et al., Molecular Cloning: A Laboratory Manual, Second Edition, Cold Spring Harbor, N.Y., 1989; and Hurrell, (Ed.), Monoclonal Hybridoma Antibodies: Techniques and Applications, CRC Press, Inc., Boca Raton, Fla., 1982).

As would be evident to one of ordinary skill in the art, polyclonal antibodies can be generated from inoculating a variety of warm-blooded animals such as horses, cows, goats, sheep, dogs, chickens, rabbits, mice, and rats with a WLDS polypeptide or a fragment thereof. The immunogenicity of a WLDS polypeptide may be increased through the use of an adjuvant, such as alum (aluminum hydroxide) or Freund's complete or incomplete adjuvant. Polypeptides useful for immunization also include fusion polypeptides, such as fusions of WLDS or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (ISA) or tetanus toxoid) for immunization.

As used herein, the term “antibodies” includes polyclonal antibodies, affinity-purified polyclonal antibodies, monoclonal antibodies, and antigen-binding fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced.

Alternative techniques for generating or selecting antibodies useful herein include in vitro exposure of lymphocytes to WLDS polypeptides, and selection of antibody display libraries in phage or similar vectors (for instance, through use of immobilized or labeled WLDS polypeptide). Genes encoding polypeptides having potential WLDS polypeptides binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides which interact with a known target which can be a protein or polypeptide, such as a ligand or receptor, a biological or synthetic macromolecule, or organic or inorganic substances. Techniques for creating and screening such random peptide display libraries are known in the art (Ladner, et al., U.S. Pat. No. 5,223,409; Ladner, et al., U.S. Pat. No. 4,946,778; Ladner, et al., U.S. Pat. No. 5,403,484 and Ladner, et al., U.S. Pat. No. 5,571,698) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.) and Pharmacia LKB Biotechnology Inc. (Piscataway, N.J.).

Embodiments of the present invention also provide for isolated and purified WLDS polynucleotide probes or primers. WLDS polynucleotide probes can be RNA or DNA. DNA can be either cDNA or genomic DNA. In general, polynucleotide probes are single or double-stranded DNA or RNA, generally synthetic oligonucleotides, but may be generated from cloned cDNA or genomic sequences and will generally comprise at least 16 nucleotides, between about 17 and 25 nucleotides, and between about 25 and 36 nucleotides. Probes and primers are generally synthetic oligonucleotides, but may be generated from cloned cDNA or genomic sequences or its complements. Analytical probes will generally be about 20 nucleotides in length, although somewhat shorter probes (14-17 nucleotides) can be used. PCR primers are at least 5 nucleotides in length, preferably 15 or more nucleotides, more preferably 20-30 nucleotides.

Probes can be labeled to provide a detectable signal, such as with an enzyme, biotin, a radionuclide, fluorophore, chemiluminescer, paramagnetic particle and the like, which are commercially available from many sources, such as Molecular Probes, Inc., Eugene, Oreg., and Amersham Corp., Arlington Heights, Ill., using techniques that are well known in the art. Techniques for developing polynucleotide probes and hybridization techniques are known in the art. (Ausubel, et al., Eds., Current Protocols in Molecular Biology, John Wiley and Sons, Inc., N.Y., 1991).

WLDS polypeptides may be used within diagnostic systems to detect axonal degeneration. The information derived from such detection methods would provide insight into the significance of WLDS polypeptides in various diseases, and can serve as diagnostic tools for diseases for which axonal degeneration are significant. Altered levels of WLDS polypeptides may be indicative of pathological conditions, as defined above.

In a basic assay, a single-stranded probe molecule is incubated with RNA, isolated from a biological sample, under conditions of temperature and ionic strength that promote base pairing between the probe and target WLDS polynucleotide. After separating unbound probe from hybridized molecules, the amount of hybrids is detected.

Well-established hybridization methods of polypeptide detection include northern analysis and dot/slot blot hybridization (Ausubel, et al., Eds., Current Protocols in Molecular Biology, John Wiley and Sons, Inc., N.Y., 1991, and Wu, et al. (Eds.), “Analysis of Gene Expression at the RNA Level,” in Methods in Gene Biotechnology, pages 225-239 (CRC Press, Inc. 1997)). Nucleic acid probes can be detectably labeled with radioisotopes such as ³²P or ³⁵S. Alternatively, WLDS polynucleotide can be detected with a nonradioactive hybridization method (Isaac (ed.), Protocols for Nucleic Acid Analysis by Nonradioactive Probes, Humana Press, Inc., 1993). Typically, nonradioactive detection is achieved by enzymatic conversion of chromogenic or chemiluminescent substrates. Illustrative nonradioactive moieties include biotin, fluorescein, and digoxigenin.

WLDS polynucleotide probes are also useful for in vivo diagnosis. As an illustration, ¹⁸F-labeled WLDS polynucleotides can be administered to a subject and visualized by positron emission tomography (Tavitian, et al., Nature Medicine, 4: 467, 1998).

Numerous diagnostic procedures take advantage of the polymerase chain reaction (PCR) to increase sensitivity of detection methods. Standard techniques for performing PCR are well-known (Mathew (Ed.), Protocols in Human Molecular Genetics, (Humana Press, Inc. 1991), White (Ed.), PCR Protocols: Current Methods and Applications (Humana Press, Inc. 1993), Cotter (Ed.), Molecular Diagnosis of Cancer (Humana Press, Inc. 1996), Hanausek and Walaszek (Eds.), Tumor Marker Protocols, (Humana Press, Inc. 1998), Lo (Ed.), Clinical Applications of PCR (Humana Press, Inc. 1998), and Meltzer (Ed.), PCR in Bioanalysis (Humana Press, Inc. 1998)).

PCR amplification products can be detected using a variety of approaches. For example, PCR products can be fractionated by gel electrophoresis, and visualized by ethidium bromide staining. Alternatively, fractionated PCR products can be transferred to a membrane, hybridized with a detectably-labeled WLDS polynucleotide probe, and examined by autoradiography. Additional alternative approaches include the use of digoxigenin-labeled deoxyribonucleic acid triphosphates to provide chemiluminescence detection, and the C-TRAK colorimetric assay.

Formulations

The WLDS polypeptides and the pharmaceutically acceptable salts and solvates thereof can be prepared in a physiologically acceptable formulation, such as in a pharmaceutically acceptable carrier medium and/or excipient, using known techniques. For example, the WLDS polypeptide can be combined with a pharmaceutically acceptable excipient to form a therapeutic composition (hereinafter WLDS composition).

Alternatively, the WLDS polynucleotide for the WLDS polypeptide can delivered in a vector for continuous administration using gene therapy techniques. The vector may be administered in a vehicle having specificity for a target site, such as a tumor.

By “pharmaceutically acceptable salt” it is meant those salts which are, within the scope of sound medical judgement, suitable for use in contact with the tissues of hosts without undue toxicity, irritation, allergic response and the like, and are commensurate with a reasonable benefit/risk ratio and effective for their intended use.

WLDS compositions may be suitable for oral, rectal, nasal, topical (including buccal and sublingual), vaginal, or parenteral (including subcutaneous, intramuscular, subcutaneous, intravenous, intradermal, intraocular, intratracheal, intracisternal, intraperitoneal, and epidural) administration.

WLDS compositions may conveniently be presented in unit dosage form and may be prepared by conventional pharmaceutical techniques. Such techniques include the step of bringing into association a WLDS polypeptide and one or more pharmaceutical carriers or excipients.

WLDS compositions suitable for oral administration may be presented as discrete units such as, but not limited to, tablets, caplets, pills or dragees capsules, or cachets, each containing a predetermined amount of one or more of the compositions; as a powder or granules; as a solution or a suspension in an aqueous liquid or a non-aqueous liquid; or as an oil-in-water liquid emulsion or a water-in-oil emulsion or as a bolus, etc.

WLDS compositions suitable for topical administration in the mouth include for example, lozenges, having the ingredients in a flavored basis, usually sucrose and acacia or tragacanth; pastilles, having a WLDS polypeptide in an inert basis such as gelatin and glycerin, or sucrose and acacia; and mouthwashes, having one or more of the compositions of the present invention administered in a suitable liquid carrier.

WLDS compositions suitable for topical administration to the skin may be presented as ointments, creams, gels, and pastes, having a WLDS polypeptide administered in a pharmaceutical acceptable carrier.

WLDS compositions for rectal administration may be presented as a suppository with a suitable base comprising, for example, cocoa butter or a salicylate.

WLDS compositions suitable for nasal administration, when the carrier is a solid, include a coarse powder having a particle size, for example, in the range of 20 to 500 microns which is administered in the manner in which snuff is taken, (i.e., by rapid inhalation through the nasal passage from a container of the powder held close up to the nose). When the carrier is a liquid (for example, a nasal spray or as nasal drops), WLDS polypeptides can be admixed in an aqueous or oily solution, and inhaled or sprayed into the nasal passage.

WLDS compositions suitable for vaginal administration may be presented as pessaries, tampons, creams, gels, pastes, foams or spray formulations containing a WLDS polypeptide and appropriate carriers.

WLDS compositions suitable for parenteral administration include aqueous and non-aqueous sterile injection solutions which may contain anti-oxidants, buffers, bacteriostats, and solutes which render the formulation isotonic with the blood of the intended recipient; and aqueous and non-aqueous sterile suspensions which may include suspending agents and thickening agents. WLDS compositions may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules, and tablets of the kind previously described above.

Pharmaceutical organic or inorganic solid or liquid carrier media suitable for enteral or parenteral administration can be used to fabricate the compositions. Gelatin, lactose, starch, magnesium stearate, talc, vegetable and animal fats and oils, gum, polyalkylene glycol, water, or other known carriers may all be suitable as carrier media.

WLDS compositions may be used as the active ingredient in combination with one or more pharmaceutically acceptable carrier mediums and/or excipients. As used herein, “pharmaceutically acceptable carrier medium” includes any and all carriers, solvents, diluents, or other liquid vehicles, dispersion or suspension aids, surface active agents, isotonic agents, thickening or emulsifying agents, preservatives, solid binders, lubricants, adjuvants, vehicles, delivery systems, disintegrants, absorbents, preservatives, surfactants, colorants, flavorants, or sweeteners and the like, as suited to the particular dosage form desired.

Additionally, WLDS compositions may be combined with pharmaceutically acceptable excipients, and, optionally, sustained-release matrices, such as biodegradable polymers, to form therapeutic compositions. A “pharmaceutically acceptable excipient” refers to a non-toxic solid, semi-solid or liquid filler, diluent, encapsulating material or formulation auxiliary of any type.

Except insofar as any conventional carrier medium is incompatible with WLDS compositions used in practicing embodiments of the invention, such as by producing any undesirable biological effect or otherwise interacting in a deleterious manner with WLDS polypeptides of the pharmaceutical composition, its use is contemplated to be within the scope of the embodiments of this invention.

When used in the above or other treatments, a therapeutically effective amount of WLDS compositions may be employed in pure form or, where such forms exist, in pharmaceutically acceptable salt. By a “therapeutically effective amount” of a WLDS polypeptide it is meant a sufficient amount of one or more of the components to treat a condition, at a reasonable benefit/risk ratio applicable to any medical treatment.

It will be understood, however, that the total daily usage of WLDS compositions will be decided by the attending physician within the scope of sound medical judgment. The specific therapeutically effective dose level for any particular host will depend upon a variety of factors, including for example, the disorder being treated and the severity of the disorder; activity of the specific composition employed; the specific composition employed, the age, body weight, general health, sex and diet of the patient; the time of administration; route of administration; rate of excretion of the specific compound employed; the duration of the treatment; drugs used in combination or coincidential with the specific composition employed; and like factors well known in the medical arts. For example, it is well within the skill of the art to start doses of WLDS compositions at levels lower than those required to achieve the desired therapeutic effect and to gradually increase the dosage until the desired effect is achieved.

WLDS compositions are preferably formulated in dosage unit form for ease of administration and uniformity of dosage. “Dosage unit form” as used herein refers to a physically discrete unit of WLDS composition appropriate for the host to be treated. Each dosage should contain the quantity of WLDS compositions calculated to produce the desired therapeutic affect either as such, or in association with the selected pharmaceutical carrier medium.

In general, the starting dose of most Phase I clinical trials is based on preclinical testing, and is usually quite conservative. A standard measure of toxicity of a drug in preclinical testing is the percentage of animals (rodents) that die because of treatment. The dose at which 10% of the animals die is known as the LD₁₀, which has in the past often correlated with the maximal-tolerated dose (MTD) in humans, adjusted for body surface area. The adjustment for body surface area includes host factors such as, for example, surface area, weight, metabolism, tissue distribution, absorption rate, and excretion rate. Thus, the standard conservative starting dose is one tenth the murine LD₁₀, although it may be even lower if other species (i.e., dogs) were more sensitive to the drug. It is anticipated that a starting dose for WLDS compositions in Phase I clinical trials in humans will be determined in this manner. (Freireich E J, et al., Cancer Chemother Rep 50: 219-244, 1966).

As stated above, a therapeutically effective dose level will depend on many factors. In addition, it is well within the skill of the art to start doses of WLDS compositions at relatively low levels, and increase the dosage until the desired effect is achieved.

WLDS compositions may be used in combination with other WLDS compositions, medicines and/or procedures for the treatment of the conditions described above.

WLDS compositions may be used with a sustained-release matrix. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

As indicated above, WLDS compositions may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically-acceptable and metabolizable lipid capable of forming liposomes can be used. The liposome can contain, in addition to WLDS compositions, stabilizers, preservatives, excipients, and the like. The preferred lipids are the phospholipids and the phosphatidyl cholines (lecithins), both natural and synthetic. Methods to form liposomes are known in the art.

### EXAMPLES

Using an in vitro model of vincristine neuropathy it can be demonstrated that toxin-induced axonal degeneration is a calcium-dependent, calpain mediated process, and that pharmacological inhibition of calpains is protective. These findings indicate that common mechanisms are involved in Wallerian degeneration and peripheral neuropathy, and have implications for understanding the pathogenesis of axonal degeneration in a number of neurological disorders.

### Example A

Materials and Methods

Dorsal Root Ganglia (DRG) Cultures

Tissue culture dishes (for example, BD Falcon™ tissue culture dishes, manufactured by BD Biosciences Discovery Labware in Bedford, Mass., United States, and commercially available from American Scientific and Industrial Supplies of Radnor, Pa., United States) of, for example, 35×10 mm are pre-coated with rat tail collagen (Type 1, available from Becton Dickinson), air dried and rehydrated with DMEM (formerly GIBCO brand, now manufactured commercially available from Invitrogen Corporation, Carlsbad, Calif., United States) overnight at room temperature and are then stored at 4° C. On the day of DRG culture, the dishes are washed twice with PBS buffer (pH 7.4), filled with 550 μl medium and pre-incubated at 37 C for at least 2 hours. Fifteen-day old embryos (E15) are removed from pregnant Sprague-Dawley rats (Charles River), and spinal cords with cervical and thoracic DRGs attached are dissected into Leibovitz L-15 medium (GIBCO). Ganglia are separated from the spinal cord, stripped of their connective tissue sheaths and roots, and are then pooled and washed twice with PBS buffer (pH 7.4). DRGs are plated (4 per dish) in culture media and incubated at 37° C. in 5% carbon dioxide for 4 hours to allow DRGs to attach to the substrate. Medium is then added to bring the total volume to 1 ml. Standard media can be, for example, MEM (GIBCO, free calcium 1.8 mM), supplemented with 1% N2 supplement (GIBCO), 7S NGF (manufactured by Alomone Labs, Jerusalem, Israel) 100 ng/ml, and 1.4 mM L-glutamine (manufactured by and commercially availabe from manufactured by and commercially available from Sigma-Aldrich Company, St. Louis, Mo., United States). Calcium-free medium is prepared in the same manner, replacing MEM with S-MEM (GIBCO).

1.1.1. Immunostaining of DRGs

At the end of the treatment period (see below) DRGs are fixed for 30 minutes with 4% paraformaldehyde. Cultures are then rinsed with 0.1M TBS buffer, and treated sequentially with 3% H₂O₂, TBS-Triton, and 4% normal goat serum (NGS), each for 30 minutes at room temperature. DRGs are incubated at 4 C overnight in monoclonal antibody to MAP5 (1:500, Sigma). After washing in TBS-Triton, DRGs are incubated for 60 minutes in biotinylated secondary antibody, rinsed with TBS and reacted with avidin-biotin complex solution (ABC; Vector Labs) for one hour. Color is generated by incubation for 10 minutes in diaminobenzidine (DAB) solution, enhanced by addition of 0.025% cobalt chloride and 0.02% nickel ammonium sulfate. Stained tissue is rinsed, air dried and cover-slipped for microscopy with Crystal/Mount (Biomeda).

1.1.2. Vincristine Neuropathy

DRGs are allowed to mature for 5 days (with a media change on day 3) creating a lush halo of neurites. This method of allowing neuritic extension to proceed before addition of a neurotoxin tests the effect of the toxin on established neurites as opposed to the effect on primary neuritic outgrowth. Thus, the in vitro paradigm is partially comparable to the clinical situation in that an “established” peripheral nervous system is exposed to a toxic agent.

After day 5 of culture, the media is changed to that containing the experimental treatment. This date is defined as treatment day 0. Cultures are monitored and imaged daily using video microscopy. Vincristine sulfate salt (Sigma) is dissolved in culture medium, aliquoted and stored at −20° C. EGTA (Sigma) is dissolved in 10 N NaOH, and diluted with ddH₂O to a stock concentration of 0.2 M. The final concentration of NaOH in DRG culture is 0.005 N, which showed no negative effects on the DRG cultures. AK 295 (Z-leu-Abu-(CH₂)₃-4-morpholinyl, gift of Dr. James Powers, Georgia Institute of Technology), is dissolved in 100% DMSO and is then diluted to its final concentration with culture medium. The final concentration of DMSO is 0.05%. Phenylmethylsulfonyl fluoride (PMSF) is manufactured by and commercially available from Sigma. Addition of EGTA, AK295, or DMSO to control cultures shows no effects on neurite growth or survival (Table 3).

After 6 days of treatment (11 days in culture) immunostained DRGs are quantitated for degree of axonal degeneration. Images of the DRGs and neurites are captured onto disk using a computerized video imaging system, and are analyzed using NIH Image version 1.61. DRG areas are calculated by tracing the outside circumference of the remaining culture halo. The length of the longest neurite of each DRG is measured from the center of the DRG to the distal end of the neurite, so that cultures without remaining neurites still have positive values. These quantitative data are subjected to ANOVA, with post-test correction for multiple comparisons.

1.1.3. Wallerian Degeneration

Five-day old cultures with extended neurites are used. Neurites are cut by excising and removing the DRG with a scalpel blade. Care should be taken to remove substantially all neurons within the DRG. At least 1 ganglion/dish was left unaxotomized to serve as a control for that group of ganglia. For experiments involving therapeutic interventions, the media is changed just prior to axotomy to media containing either EGTA (2 mM) or AK295 (50 μM). Cultures are observed daily for 72 hours after axotomy, at which time they are fixed and immunostained.

Stained cultures are scored blindly for degree of axonal degeneration. For axotomized neurites, as opposed to those treated with vincristine, degeneration is considered an all or none phenomenon. Any fibers with interruptions along their length are scored as degenerated. The number of surviving fibers in each DRG are counted under 200× magnification, and the DRG is given a survival score of 0-3: 0 =no fibers remaining, 1=1-4 fibers remaining, 2=5-9 fibers remaining, 3=10 or more fibers remaining. The scores are subjected to ANOVA, with post-test correction for multiple comparisons.

Results

1.1.4. Vincristine Induced Axonal Degeneration

To determine the sensitivity of cultured DRG neurites to vincristine, drug concentrations of 0.01 to 4 μM are added to 5 day-old cultures. Axonal degeneration occurs very quickly (within 1-3 days) at concentrations 0.05 μM. At 0.01 μM, the process of axonal degeneration is relatively slow, allowing for discrimination of changes between treated and untreated cultures over time.

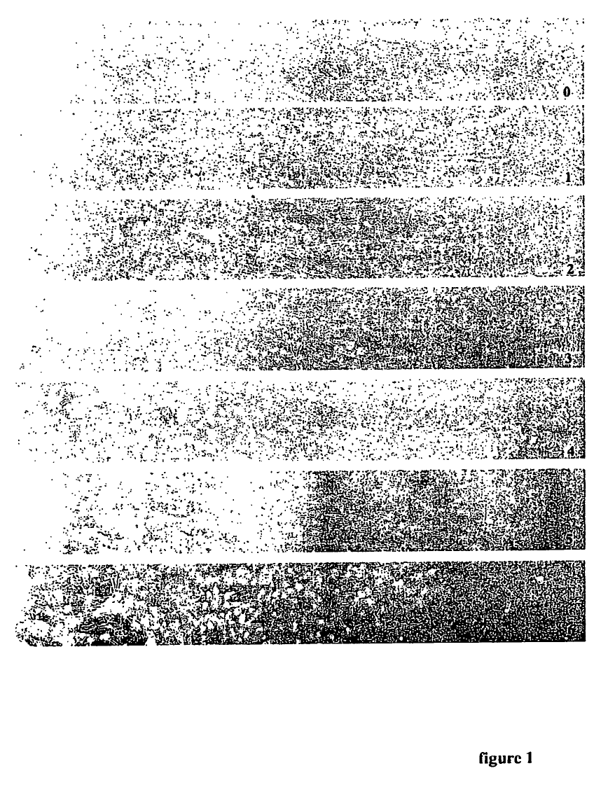

Signs of degeneration including axonal beading and segmentation are identified as early as twelve hours after exposure to vincristine. At 24 to 48 hours of exposure, the distal portions of axons show significant pathological changes. Degeneration proceeds in a distal to proximal pattern along neuritic bundles until fibers are completely replaced by axonal debris at 5 to 6 days (FIG. 1). The neuronal cell bodies also show changes, with the DRG becoming smaller over the 6 day time course. These changes are not quantified herein.

Vincristine exposure produces a progressive reduction in the area of the DRG neuritic halo (Table 4). At day 0, the DRG area is 28.98±1.76 mm² and after three days exposure to vincristine is reduced by 76.6% to 6.78±1.06 mm² (p<0.01). At 4 days exposure the DRG area is 2.45±0.74 mm², and at 6 days 0.16±0.61 mm², representing respectively a 91.5% and a 97.9% reduction in area. In the control group, axons continued to grow during the experimental period, increasing to 33.62±2.98 mm² on day 3, 41.94±1.64 mm² on day 4, and 45.14±4.35 mm² on day 6. At day 6, the area of DRG halo is 155.8% larger than at treatment day 0.

Changes in the lengths of axons reflect those seen in measurements of DRG areas (Table 4). Vincristine exposure results in a 57.6% reduction in length of the longest axons at day 3 (3.82±0.11 mm to 1.62±0.19 mm). At days 4 and 6 of vincristine exposure, axon length decreases to 0.91±0.19 mm (76.2%) and 0.37±0.06 mm (90.3%), respectively (p<0.01 for all time points). In contrast, axonal lengths in the control group increase by 130.9% of that at day 0 during the six-day period.

1.1.4.1.1.1. Protection Against Vincristine Induced Axonal Degeneration

To test the role of extra-cellular free calcium in vincristine-induced axonal degeneration, DRGs are exposed to vincristine in standard media containing EGTA, in calcium-free media, or in calcium-free media with EGTA. These strategies provide significant protection against axonal degeneration by measures of either DRG area or axonal lengths (Table 4). EGTA at a concentration of ≦1 mM is ineffective (not shown), while 2 mM is as effective as calcium-free media. There is also an additive effect of using both EGTA and calcium-free media when measured at the 6 day time point (Table 4).

The neuroprotective effects of a low calcium environment lead us to test whether calpains are also important in the pathogenesis of vincristine neuropathy. Addition of the experimental calpain inhibitor AK295 is effective in preventing axonal degeneration at doses of either 50 μM (Table 4) or 10 μM (not shown), and is ineffective at 1 μM (not shown). The neuroprotective effect of AK295 is equal to that of either 2 mM EGTA or calcium-free media.

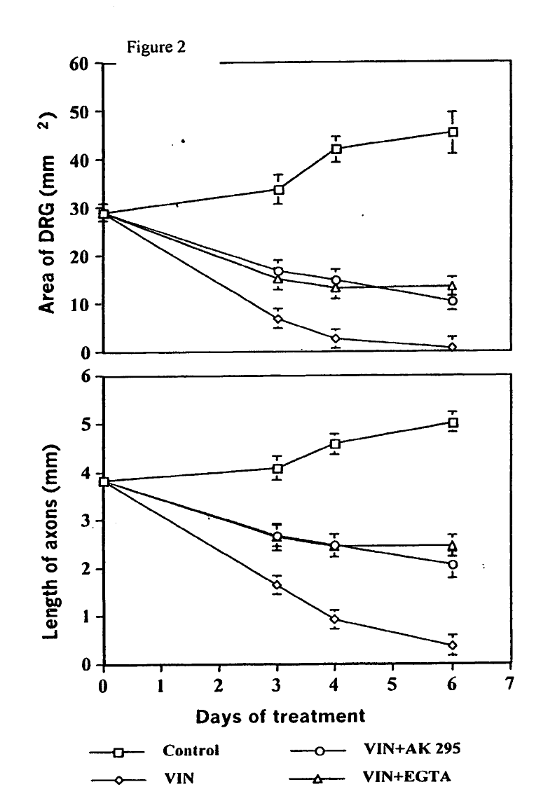

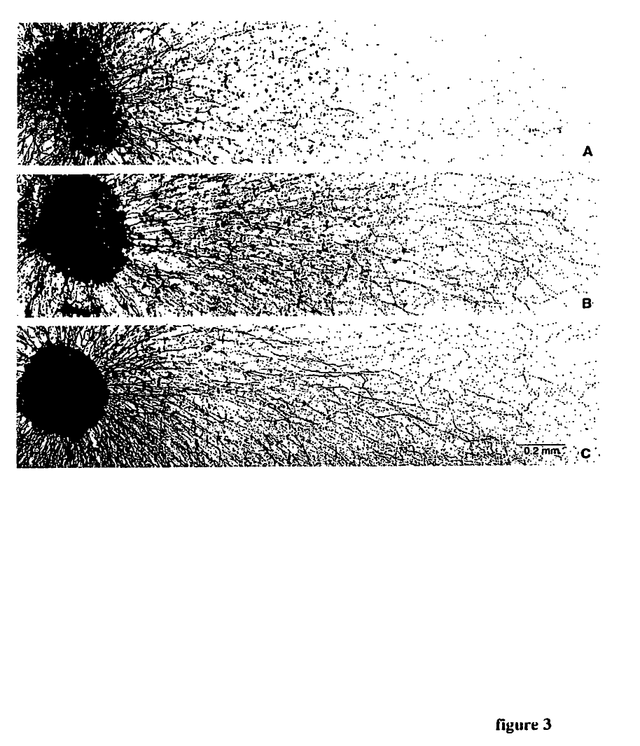

The low calcium environment or treatment with AK295 does not provide complete protection against axonal degeneration. When the quantitative measures are viewed over the entire 6-day time course, a graded effect is revealed. Neurites exposed to vincristine but maintained in the neuroprotective media stop growing (FIG. 2), and show morphologic changes typical of axonal degeneration (FIG. 3). These changes are, however, both qualitatively and quantitatively less severe than those seen in cultures treated with vincristine alone.

To ensure that the protective effect AK295 is not a non-specific characteristic of protease inhibitors we study whether the serine protease inhibitor PMSF, which is not an inhibitor of calpains, could protect against axonal degeneration in this model. PMSF shows no protective effects (Table 4).

1.1.5. Wallerian Degeneration

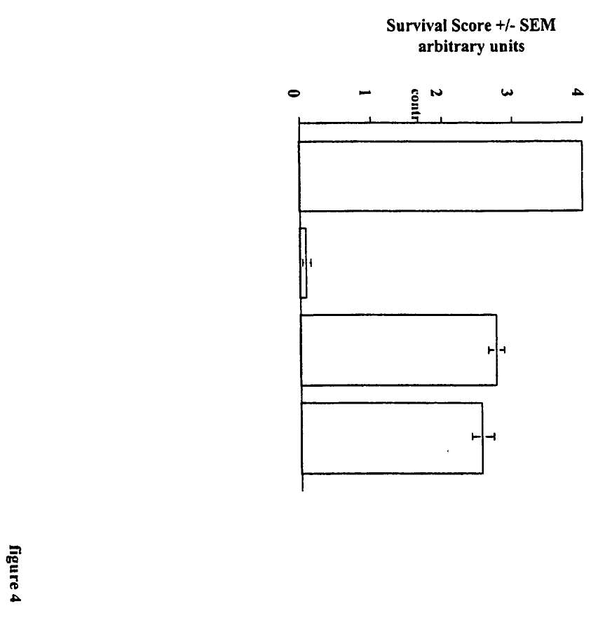

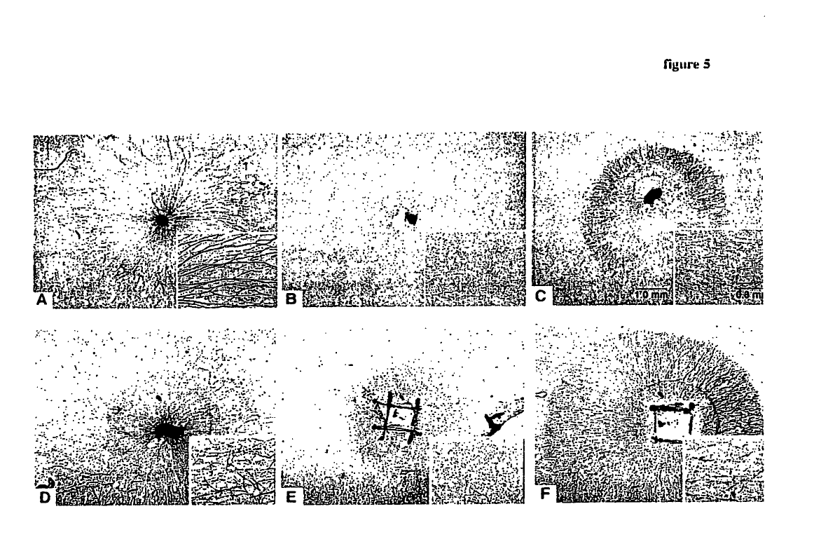

Axotomized neurites are completely degenerated by 72 hours after transection. Addition of EGTA at the time of axotomy provides significant protection against Wallcrian degeneration (FIG. 4). Addition of the calpain inhibitor AK295 is as protective as EGTA. A direct comparison of the protective effects of AK295 in vincristine neuropathy and Wallerian degeneration is demonstrated in FIG. 5. The preservation of axons with AK295 is not a subtle finding, and is seen easily in these low power photomicrographs.

Discussion

These data demonstrate that like axotomy-induced Wallerian degeneration, axonal degeneration in vincristine neuropathy is both a calcium-dependent and calpain-mediated event. This in vitro model of vincristine neuropathy provides an experimental system with a high degree of similarity to the human condition. First, these tests are done with primary sensory neurons and axons and not neuronal cell lines. Second, the measures of neurotoxicity and neuroprotection are on the degeneration of developed axons as opposed to inhibition of initial axon outgrowth. Third, the toxic neuropathy progresses from distal to proximal, as is seen in human neuropathies.

The pathogenesis of vincristine neuropathy is thought to be a consequence of its primary antineoplastic function as a mitotic spindle inhibitor. Experimental data suggest that vincristine alters the structure of axonal microtubules, leading to abnormalities in fast axonal transport. Dysfunctional axonal transport is a major theory for the pathogenesis of a variety of toxic neuropathies, and is considered a causative factor in the case of vincristine. Intoxication of laboratory animals, or direct exposure of nerves to vincristine has been reported to cause alterations in the structure and shape of axonal microtubules, shortening of microtubule lengths, or changes in microtubule distribution within the axon. Neurofilamentous axonal swellings, as is seen with hexacarbon and other intoxications, have also been reported with vincristine. These changes are believed to provide the pathological substrate for altered axonal transport, which may affect the delivery of nutritive substances to the axon from the cell body. Changes in axonal transport profiles, including both slowing and acceleration of transport peaks, has been demonstrated in vincristine-intoxicated cats.

If disruption of normal axonal transport is the cause of vincristine neuropathy, it is unclear why a low calcium environment or inhibition of calpains is protective. One possibility is that the axonal transport hypothesis is incorrect. However, the cumulative evidence supporting microtubular and transport abnormalities is strong. We believe that the neuroprotective effects of low calcium and calpain inhibition reflect a final common pathway of axonal degeneration that may become active following a variety of axonal or neuronal insults. Calcium entry into neurons and axons is a common feature of experimental models of acute and chronic neurologic injury. These include Wallerian degeneration, nerve hypoxia, glutamate-induced excitotoxicity, and other toxic neuropathies. Calpain activation has also been implicated in the pathogenesis of a wide variety of neurologic disorders such as stroke, head and spinal cord trauma, and even Alzheimer disease. It is believed that elevated intracellular calcium in injured cells leads to pathologic activation of calpains, and results in neuronal/axonal degeneration. In the case of vincristine neuropathy, altered axonal transport may lead to elevations of intracellular calcium and activation of calpains through metabolic compromise, alteration of calcium conductance, or loss of membrane integrity.

The therapeutic potential of calpain inhibitors is based on their effectiveness in preventing axonal degeneration, but may be somewhat limited by their inherent cellular toxicity. Calpains are ubiquitous cytosolic enzymes that are putatively involved in a number of normal cellular functions. Certainly, chronic calpain inhibition has the potential for interfering with these functions. Previous uses of AK295 have been in acute neurologic injuries, where drug exposure has been limited to single or short term dosing. In our DRG cultures we find that chronic calpain inhibition is neither toxic to neurites, nor does it affect their normal growth.

The results of these tests not only provide new insight into the pathogenesis of vincristine neuropathy, but have implications for understanding the general mechanisms underlying axonal degeneration. It is demonstrated herein that a common pathway involving calcium and calpains leads to axonal degeneration in two different models of axonal injury, Wallerian degeneration and vincristine exposure. These findings secure the link between Wallerian and Wallerian-like degeneration by demonstrating common pathophysiology. Neuroprotective effects of calpain inhibition in our model of vincristine neuropathy are pertinent for a wide variety of neurological disorders where axonal degeneration is prominent, including disorders of the PNS and CNS. Calpain inhibition may be a reasonable strategy for preventing axonal degeneration and preserving neurologic function in acute injuries such as for example, but not limited to, stroke and trauma, or in chronic disorders such as for example, but not limited to, diabetes mellitus, hereditary neuropathies, or multiple sclerosis.

TABLE 3
Lack of effect of treatment modalities
on DRG area and neuritic length

| | Area (mm²) | Length (mm) |
|---|---|---|
| Control (15)* | 45.14 ± 4.35 | 5.00 ± 0.22 |
| EGTA (2 mM) (7) | 44.40 ± 2.74 | 4.74 ± 0.18 |
| AK 295 (50 M) (11) | 49.18 ± 2.69 | 4.80 ± 0.16 |
| DMSO (0.05%) (9) | 40.80 ± 2.33 | 4.48 ± 0.15 |

Treatments were added to 5-day old cultures.
Quantitative measurements were made after 6 days of treatment.
Values are mean ± SEM. p > 0.05, (NS) for all groups.
*(n)

TABLE 4
DRG areas and neuritic lengths in control cultures and treated cultures

| | | | Day3 | | Day4 | | Day6 | |
|---|---|---|---|---|---|---|---|---|
| | Area | Length | Area | Length | Area | Length | Area | Length |
| Control | 28.98 ± 1.76 (13) | 3.82 ± 0.11 | 33.62 ± 2.98 (12) | 4.07 ± 0.25 | 33.62 ± 2.98 (10) | 4.56 ± 0.15 | 45.14 ± 4.35 (15) | 5.00 ± 0.22 |
| Vin | | | 6.78 ± 1.06 (17) | 1.62 ± 0.19 | 2.45 ± 0.74 (13) | 0.91 ± 0.19 | 0.61 ± 0.21 (48) | 0.37 ± 0.06 |
| | | | 16.61 ± 1.18 (18) | 2.65 ± 0.11 | 14.77 ± 2.19 (14) | 2.45 ± 0.25 | 10.34 ± 2.14 (12) | 2.05 ± 0.25 |
| Vin + | | | 15.05 ± 1.78# (12) | 2.62 ± 0.17 | 12.97 ± 0.94 | 2.44 ± 0.13 | 13.36 ± 1.04 (14) | 2.43 ± 0.07 |
| EGTA* | | | | | (14) | | | |
| Vin + | | | | | | | 14.90 ± 3.22 (3) | 2.47 ± 0.29 |
| Ca⁺⁺− | | | | | | | | |
| Free* | | | | | | | | |
| Vin + | | | | | | | 21.77 ± 1.74 (11) | 3.08 ± 0.14 |
| EGTA + | | | | | | | | |
| Ca⁺⁺ − | | | | | | | | |
| Free** | | | | | | | | |
| Vin + PMSF | | | | | | | 0.73 ± 0.16 (43) | 0.55 ± 0.06 |
| (50 μM) | | | | | | | (NS) | (NS) |

Area in mm², length in mm. Values are mean ± SEM.
*p < 0.01,
**p < 0.001,
# p < 0.05, comparison to vincristine treatment alone.
Drug concentrations are: Vin 0.01 μM, AK295 50 μM, EGTA 2 mM. Numbers in parentheses are the number of experiments performed.

### Example B

Materials and Methods

Dorsal Root Ganglion (DRG) Cultures

Tissue culture dishes (BD Falcon™) of 35×10 mm were pre-coated with rat tail collagen (type 1, Becton Dickinson), air dried and rehydrated with DMEM (GIBCO) overnight at room temperature and then stored at 4° C. On the day of DRG culture, the dishes were washed twice with PBS buffer (pH 7.4), filled with 550 μl medium and pre-incubated at 37 C for at least 2 hours. DRGs were dissected from newborn mice (C57BL/6, Charles River and WLD^{S}, breeding colony maintained at the Emory University School of Medicine). Ganglia were transferred into L-15 medium (GIBCO), separated from roots and connective tissue sheaths, pooled and washed twice with PBS buffer (pH 7.4). DRGs were plated (5 per dish) in culture media (MEM), supplemented with 1% N2 (GIBCO), 7S NGF 100 ng/ml, and 1.4 mM L-glutamine (Sigma) and incubated at 37° C. in 5% carbon dioxide.

Vincristine Neuropathy

Vincristine sulfate salt (Sigma) was dissolved in culture medium, aliquoted and stored at −20° C. DRGs were allowed to mature for 5 days to create a lush halo of neurites. This method of allowing neuritic extension to proceed before addition of vincristine tests the effect of the vincristine on established neurites as opposed to the effect on primary neuritic outgrowth. After day 5 of culture, the media was changed to that containing vincristine. This date was defined as treatment day 0. Based on the results of pilot experiments, four concentrations of vincristine were chosen for investigation: 0.01, 0.02, 0.05 and 0.1 μM. Cultures were monitored and imaged on day 0, day 4, day 8 and day 10 of treatment using video microscopy.

Imaging and Statistic Analysis

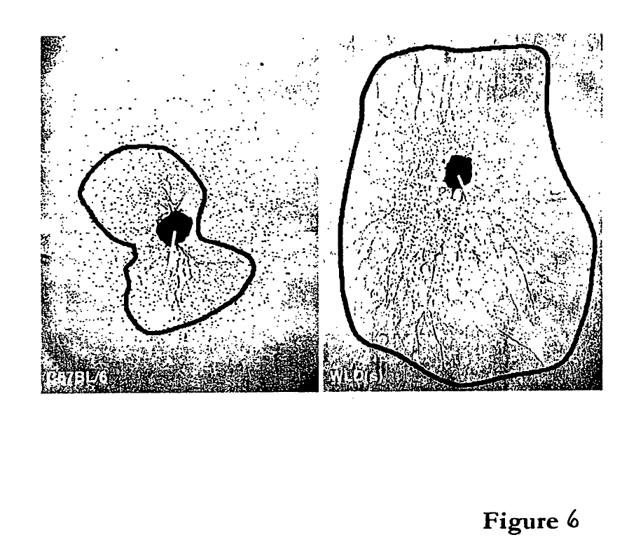

Images of the DRGs and neurites were captured onto disk using a computerized video imaging system, and analyzed using NIH Image version 1.61. Cultures were examined and measured on the initial day of vincristine exposure (day 0), as well as after 4, 8 and 10 days of exposure. Measures of axonal survival were the length of longest remaining axon, and the area of remaining DRG halo, as determined by serial phase-contrast video microscopy (FIG. 6). The axonal length was measured from the center of the DRG to the visible distal end of the axon. Halo areas were calculated by tracing the outside circumference of the remaining culture halo. Since there was variability in the physical characteristics of individual cultures, each DRG served as its own control by normalizing data at days 4, 8, and 10 to the condition before vincristine exposure. Data were thus analyzed as percent of day 0. These normalized data were subjected to ANOVA, with post-test correction for multiple comparisons.

Results:

Dose-Dependent Response of Wld^{S} and C57BL/6 Axons to Vincristine Exposure.

Morphological changes associated with vincristine exposure were first evident as beading along the distal parts of axons, followed by segmentation and then disappearance of the axons. These changes progressed in a distal to proximal pattern resulting in gradual reduction in axonal length and area of the DRG halo. Higher doses of vincristine resulted in more rapid rates of axonal degeneration in cultures from both C57BL/6 and Wld^{S} mice.

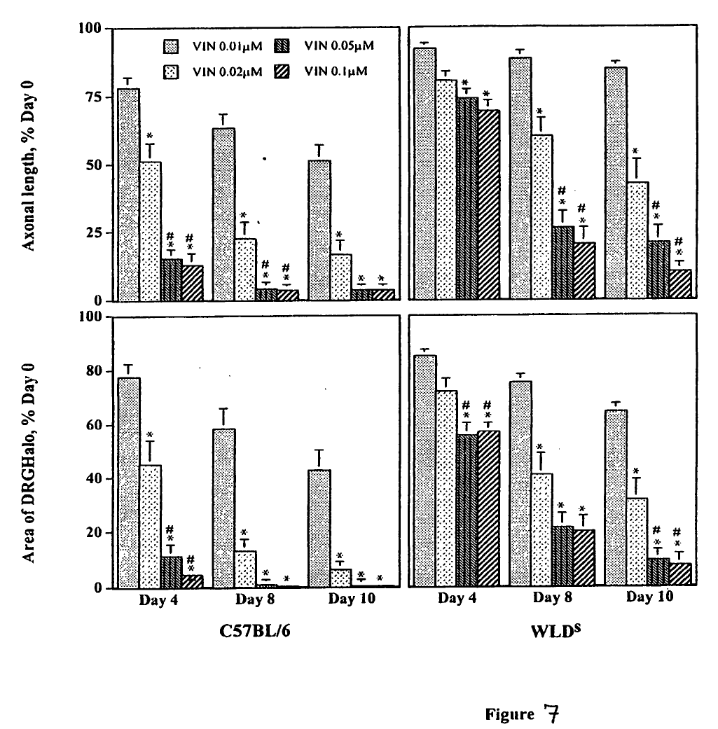

Changes in axonal lengths and halo areas in response to the 4 doses of vincristine are graphed in FIG. 7. These graphs demonstrate the clear dose dependence of vincristine exposure on axonal degeneration, and suggest the relative resistance of Wld^{S} axons (see below). In C57BL/6, the lengths of longest axons at day 10 when compared to day 0 were 50.99±5.97% when exposed to 0.01 μM, and 3.56±0.62% when exposed to 0.1 μM. The corresponding halo areas were 42.86±7.30% and 0.45±0.09%, respectively. In general, the two measures of toxicity, axonal length and area of the halo, showed good correspondence. Statistical comparisons demonstrated differences among all groups for C57BL/6 cultures, except at the highest doses where complete degeneration occurred early in the time course.

In Wld^{S} the length of longest axons at day 10 when compared to day 0 were 84.33±2.54% when exposed to 0.01 μM, and 10.17±3.22% when exposed to 0.1 μM. The corresponding halo areas were 64.75±2.62% and 7.92±3.83%, respectively. Interestingly, statistical differences were not found between the 0.01 and 0.02 μM groups at the 4 day time point, reflecting the resistance of Wld^{S} axons to degeneration. At 8 and 10 days of exposure, however, a dose effect could be demonstrated. As in the C57BL/6 cultures, no differences were found either in axon length or DRG area when comparing the two highest doses.

Axons of Wld^{S} are Resistant to Vincristine

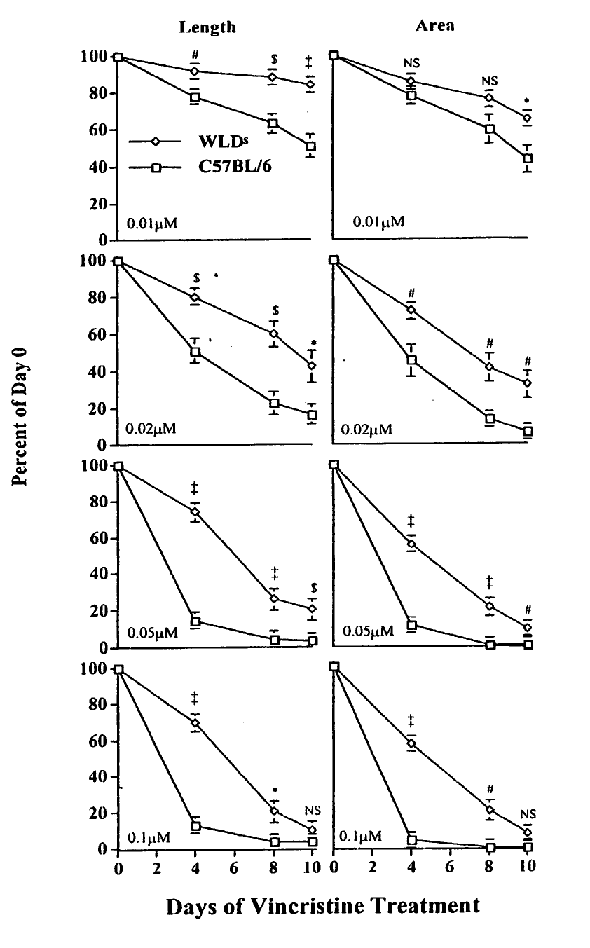

Direct comparisons of vincristine-induced axonal degeneration in C57BL/6 and Wld^{S} cultured neurites are shown in Table 5 and FIG. 8. Table 5 shows the comparisons of axonal lengths and halo areas between C57BL/6 and Wld^{S}. Note that there were no differences between the cultures at the time of vincristine exposure (day 0). Except for the lowest dose (0.01 μM) at the earliest evaluation time (4 days), statistical differences were found in all treatment groups. The most robust differences were found at the later time points (days 8 and 10), reflecting the sensitivity over time of the C57BL/6 axons, and the relative resistance of the Wld^{S} axons.

FIG. 3 compares graphically the normalized data from C57BL6 and Wld^{S} axons. Note the early divergence of the lines and the protective effect at all doses. Again, Wld^{S} neurites exposed to high doses for short time periods (0.05 and 0.1 μM for 4 days) showed remarkable resistance to axonal degeneration as compared to wild type neurites.

Discussion

These experimental studies on cultured DRG neurites demonstrate that the Wld^{S} mutation that slows Wallerian degeneration after axotomy, also provides protection against vincristine-induced axonal degeneration. The findings support a mechanistic link between these two types of axonal injury, suggesting that both pathophysiologic and therapeutic studies on Wallerian degeneration may be relevant to a variety other neurological disorders. Vincristine neurotoxicity provides a good model for non-traumatic neuropathy because it is clinically relevant (peripheral neuropathy associated with axonal degeneration is the major dose-limiting side effect of vincristine), and is faithfully reproduced in our in vitro model, causing a distal-to-proximal “dying back” neuropathy as is seen in humans.

We acknowledge that genetic background may play an important role in the susceptibility of rodent axons to injury and degeneration, and we cannot fully discount the possibility that a genetic difference between strains other than the Wld^{S} mutation may have contributed to our findings. However, the C57BL/6 is the parent strain for the Wld^{S}, and is so closely genetically related that tissue grafts between C57BL/6 and Wld^{S} survive without immunosuppression, suggesting immunologic identity.

Whether the Wld^{S} mutation only slowed down the process of vincristine-induced axonal degeneration, or provided resistance that could be measured functionally as true neuroprotection has not yet been determined. However, there is evidence from previous work that suggests the Wld^{S} mutation provides functional neuroprotection. Transected but structurally intact nerves in Wld^{S} are able to support action potentials as well as axonal transport of proteins. Recovery from intoxication is the true test of the protective potential of this mutation, and studies are underway to determine whether toxin-exposed neurites may indefinitely resist degeneration.

It is clear that the mechanisms of axonal degeneration may be different from those involved in death of the perikaryon. Using the Wld^{S} mouse model, it has been demonstrated (C: this was done by another author) that Wld^{S} sympathetic neurons deprived of NGF undergo the normal sequence of apoptosis, whereas their neurites remained structurally intact. Comparing perikaryal and axonal degeneration in cultures of normal neurons showed that perikaryal degeneration in response to neurotrophin deprivation involved activation of caspases, whereas axonal degeneration in the same neurons did not. There are also situations where preventing perikaryal apoptosis does not provide protection for the axon. Overexpression of the anti-apoptotic Bcl-2 protein protects neuronal cell bodies from degeneration but not their axons. This phenomenon has been shown in axotomized retinal ganglion cells and in the pmn mouse model of motor neuron disease. Thus, a cellular “program” for axonal death likely exists that is distinct from those involved in neuronal death. Since axonal degeneration is such an important feature of neurologic disease and dysfunction, delineating this program is essential for providing for neuroprotection in a variety of disorders including stroke, head trauma, spinal cord injury, multiple sclerosis, and peripheral neuropathy.

The pathophysiology of axonal degeneration shares common features in a variety of disorders. In experimental injury models of PNS and CNS axons including axotomy, blunt trauma, and hypoxia/ischemia, elevations of intracellular calcium are required for axonal degeneration. Increases in calcium lead to activation of calpains that are involved in proteolysis of the axonal cytoskeleton, the morphologic and biochemical hallmark of axonal degeneration. Reduction of cellular calcium or inhibition of calpains are protective in all of these models of axonal degeneration. We compared experimental vincristine neuropathy to Wallerian degeneration and demonstrated that both are calcium-dependent, calpain-mediated processes.

These new data in the Wld^{S} mouse add further support for the hypothesis that common mechanisms are involved these two forms of axonal degeneration. In addition, these findings suggest that axonal degeneration, as opposed to being a passive process of “withering” of an unsupported cellular extension, is more likely a programmed event that can be disrupted by mutation of a single gene. Even though the Wld^{S} gene has not yet been identified, clues to the mechanism of delayed Wallerian degeneration are that axonal neurofilaments from the Wld^{S} are relatively resistant to calcium-mediated degradation and specifically to calpain.

A further implication of these data relates to the fact that vincristine neuropathy, like other neuropathic disorders, is a slowly evolving process, making it amenable to early detection, treatment, and prevention. Unlike axotomy-induced axonal degeneration, where rapid degradation and removal of the distal nerve stump may be preferable for initiation of the regenerative process, axonal degeneration in slowly evolving neuropathies is potentially preventable by therapeutic strategies that preserve both structure and function. Providing resistance to axonal degeneration, thorough identification and manipulation of the Wld^{S} protein, may lead to exciting new treatment strategies for a number of neuropathic and neurodegenerative disorders.

TABLE 5
Comparisons of Vincristine-induced Axonal Degeneration in C57BL/6 and
Wld^{S} Cultured Neurites
1.1.5.2.Length of Longest Neurite

| | | Days after Vincristine treatment | | | |
|---|---|---|---|---|---|
| Vincristine | | Day 0 | Day 4 | Day 8 | Day 10 |
| (μM) | DRG (n) | length of neurite (mean ± SEM, mm) | | | |
| 0.01 | C57BL/6 (14) | 2.28 ± 0.14 | 1.77 ± 0.12 | 1.46 ± 0.15 | 1.22 ± 0.17 |
| | Wld^{S} (12) | 2.18 ± 0.11 | 1.99 ± 0.08 | 1.91 ± 0.09 | 1.83 ± 0.10 |
| | p value | 0.592 | 0.1573 | 0.0240 | 0.0069 |
| 0.02 | C57BL/6 (10) | 2.36 ± 0.24 | 1.28 ± 0.22 | 0.53 ± 0.16 | 0.41 ± 0.13 |
| | Wld^{S} (12) | 2.26 ± 0.14 | 1.83 ± 0.14 | 1.38 ± 0.18 | 1.02 ± 0.23 |
| | p value | 0.7337 | 0.045 | 0.0021 | 0.00391 |
| 0.05 | C57BL/6 (20) | 2.14 ± 0.14 | 0.32 ± 0.08 | 0.09 ± 0.01 | 0.07 ± 0.01 |
| | Wld^{S} (12) | 2.05 ± 0.09 | 1.51 ± 0.09 | 0.55 ± 0.13 | 0.42 ± 0.14 |
| | p value | 0.6346 | <0.0001 | <0.0001 | 0.0023 |
| 0.1 | C57BL/6 (12) | 2.33 ± 0.13 | 0.30 ± 0.09 | 0.08 ± 0.01 | 0.08 ± 0.01 |
| | Wld^{S} (12) | 2.34 ± 0.12 | 1.62 ± 0.09 | 0.46 ± 0.15 | 0.23 ± 0.06 |
| | p value | 0.9493 | <0.0001 | 0.0145 | 0.0227 |

1.1.5.3.Area of DRG Halo

| | | Days after Vincristine treatment | | | |
|---|---|---|---|---|---|
| Vincristine | | Day 0 | Day 4 | Day 8 | Day 10 |
| (μM) | DRG (n) | Area of DRG (mean ± SEM, mm²) | | | |
| 0.01 | C57BL/6 (14) | 9.36 ± 0.80 | 7.34 ± 0.78 | 5.79 ± 0.86 | 4.42 ± 0.85 |
| | Wld^{S} (12) | 8.63 ± 0.68 | 7.27 ± 0.51 | 6.50 ± 0.56 | 5.60 ± 0.51 |
| | p value | 0.5023 | 0.9429 | 0.5156 | 0.2627 |
| 0.02 | C57BL/6 (10) | 8.80 ± 1.45 | 4.56 ± 1.10 | 1.14 ± 0.49 | 0.56 ± 0.27 |
| | Wld^{S} (12) | 8.21 ± 0.9 | 5.95 ± 0.72 | 3.39 ± 0.64 | 2.53 ± 0.59 |
| | p value | 0.7236 | 0.2876 | 0.0138 | 0.0107 |
| 0.05 | C57BL/6 (20) | 9.47 ± 1.01 | 1.20 ± 0.47 | 0.05 ± 0.01 | 0.04 ± 0.01 |
| | Wld^{S} (12) | 7.61 ± 0.67 | 4.17 ± 0.45 | 1.63 ± 0.48 | 0.80 ± 0.36 |
| | p value | 0.1991 | 0.0002 | 0.0001 | 0.0103 |
| 0.1 | C57BL/6 (12) | 10.49 ± 0.76 | 0.46 ± 0.19 | 0.04 ± 0.01 | 0.04 ± 0.01 |
| | Wld^{S} (12) | 9.60 ± 1.66 | 5.30 ± 0.86 | 2.00 ± 0.66 | 0.59 ± 0.28 |
| | p value | 0.6283 | <0.0001 | 0.0071 | 0.0657 |

### Example C

We demonstrate that the biological potential of this mutation for modifying the response to neurologic injury extends to the slowly progressive axonal degeneration seen in a culture model of toxic peripheral neuropathy. Direct comparisons of cultured sensory axons from wild type and Wld^{S} mice exposed to varying doses of vincristine demonstrated that Wld^{S} axons remained structurally intact when wild type axons had degenerated. This observation provided evidence of a pathophysiological link between axotomy-induced Wallerian degeneration and a slowly progressive peripheral neuropathy, but also raised the important question of whether the Wld^{S} mutation may be neuroprotective. Is it possible that this mouse may be resistant to neurological disorders characterized by axonal degeneration, and could the neuroprotective phenotype be transferred through introduction of the Wld^{S} mutant gene?

In order to establish that the Wld^{S} mutation and its protein have the potential for providing protection against neurological disease, three criteria must be met. First, it is necessary to show that structurally intact Wld^{S} axons are truly alive and not merely “tombstones” without physiological function. Second, it must be proven that the mutation and protein found in the Wld^{S} mouse are together responsible for the Wld^{S} phenotype. Third, it must be demonstrated that the neuroprotective phenotype can be transferred to wild type neurons through introduction of the Wld^{S} gene. Here we provide evidence for all three measures and suggest that the introduction of the Wld^{S} gene may provide a novel approach to prevention or treatment of axonal degeneration seen in peripheral neuropathy and possibly other neurological disorders.

Methods:

Wld^{S} mice were obtained from a colony maintained at Emory University. C57BL/6 (wild-type) mice and Sprague Dawley rats were obtained from Jackson Labs.

1.2. Preparation of Recombinant Adenovirus

Total RNA and then mRNA were isolated (Oligtex mRNA mini Kit, Qiagen) from the brain of a 4-week-old Wld^{S} mouse. The Wld^{S} mutant gene (Ufd2/D4 Colele) was amplified from cDNA by PCR (pfu polymerase, Stratagene) using primers designed from the published sequence: 5′-TTA TTA GTC GAC ATG GAG GAG CTG AGC-3′, and 5′-TGA TGA ATT CTC ACA GAG TGG AAT GGT T-3′. The amplified 10.1 kb product was gel purified, digested with SalI/EcoRI, and subcloned into the pCI/IRES vector to generate pCI/IRES/Wld^{S}. The 3.7 kb CMV/Wld^{S}/IRES/GFP DNA fragment was released from the vector by digestion with BglII/ClaI, and the fragment was subcloned into the adenovirus transfer vector pAdLink. Adenovirus was generated as previously described. Briefly, the modified transfer vector was linearized with NheI for preparation of the recombinant adenovirus. One microgram of linearized transfer DNA (pAdLink. 1 CMV/Wld^{S}/IRES/GFP) was mixed with 1 μg of ClaI-linearized adenovirus backbone DNA and cotransfected into sub-confluent HEK293A cells using LipofectAMINE reagent (GIBCO/BRL). The transfected 293A cells were trypsinized and diluted with 293A cells, seeded into the 96-well tissue culture plates, and kept at 37 C, 5% CO₂ for several days. Recombinant adenovirus/Wld^{S}/IRES/GFP was selected by checking GFP expression. Expression of the Wld^{S} protein was confirmed by western blot (see below). Recombinant adenoviruses were purified from wild type viruses by the limiting dilution method. Adenovirus titers were determined by TCID-50 (Tissue Culture Infectious Dose).

The Wld^{S} gene was introduced into rat DRG cells by replacement of standard media with media containing recombinant adenovirus (10⁹ particles/cc) expressing either the lacZ gene (control) or the Wld^{S} gene. Cultures had extended neurites for 6 days before introduction of the transgene. Transgene expression, indicated by GFP fluorescence, was monitored by fluorescence microscopy. Neurons and Schwann cells were bright by 24 hours after infection; axons showed GFP fluorescence at 48 to 72 hours.

Production of Antibody

The Wld^{S} polyclonal antibody was produced in a New Zealand rabbit using standard protocols. The immunogen was a peptide sequence (YLVPDLVQEYTEK) unique to the Wld^{S} mutant protein conjugated to thyroglobulin. Rabbit serum was affinity purified against its parent peptide, and was tested for recognition of the Wld^{S} protein by western blot. Positive controls were HEK293A or DRG cells infected with adenovirus expressing the Wld^{S} protein. Negative controls were cells infected with adenovirus expressing lacZ. Specificity was determined by preadsorption of the primary antibody with parent peptide (0.1 mg/cc). Standard protocols were used for western blotting and immunocytochemistry, as previously described. A monoclonal antibody to MAP-5 (Boehringer-Mannheim) identified axons for colocalization studies. Immunofluorescent images were captured using a Zeiss 510 laser confocal microscope.

1.3. Dorsal Root Ganglia (DRG) Cultures

The methods for DRG culture have been previously described. Cultures were generated from either newborn C57BL/6 or Wld^{S} mice, or from E15 Sprague Dawley rats. Standard media was MEM (GIBCO), supplemented with 1% N2 supplement (GIBCO), 7S NGF (Alomone Labs, Jerusalem, Israel) 100 ng/ml, and 1.4 mM L-glutamine (Sigma). Suppression of Schwann cell growth was accomplished by addition of fluorodeoxyuridine and uridine (both at a dose of 10⁻⁶M, Sigma) on days 2-5. Prior to any manipulation (ie., exposure to vincristine or infection with adenovirus) cultures were grown for either 5 (mouse) or 6 (rat) days to allow for a rich halo of neurites.

Vincristine Neuropathy

The model of vincristine neuropathy in rat and mouse DRG cultures has been previously described. Cultures from C57BL/6 and Wld^{S} were allowed 5 days of growth in standard media, after which the media was exchanged for that containing 0.05 μM vincristine sulfate (Sigma). After 24 hours of vincristine exposure, vincristine was removed through media exchange, and cultures were observed and measured by phase-contrast video microscopy for an additional 19 days (total 20 days after exposure). Cultures were measured for length of longest axon and area of the DRG halo at days 4, 8, 12, 16 and 20. Lengths were measured from the center of the DRG to the visible distal end of the axon, and areas were calculated by tracing the circumference crested by connecting the tips of the remaining axons. Length and area data were normalized by allowing each DRG to serve as its own control, and measurements were calculated as percent change as compared to day 0 (day of vincristine exposure). Normalized data were subjected to ANOVA, with post-test correction for multiple comparisons. Comparisons were made between quantitative data for C57BL/6 and Wld^{S} axons. For assessment of growth, day 4 observations in Wld^{S} were compared to all succeeding days using ANOVA for repeated measures.

Rat DRG cultures were used to test whether expression of the Wld^{S} transgene provides for resistance to vincristine-induced axonal degeneration. E15 DRG were grown in culture for 6 days and then exposed to adenovirus (with or without the Wld^{S}transgene) as described above. On the next day, cultures were treated with 0.01 μM vincristine. Cultures were observed for 10 days during continuous vincristine exposure, with phase contrast images recorded at days 0, 4, 8, and 10. Length and area measurements were performed and analyzed as described above for mouse cultures.

Results:

### 1.3.1. Vincristine Withdrawal in Mouse Cultures

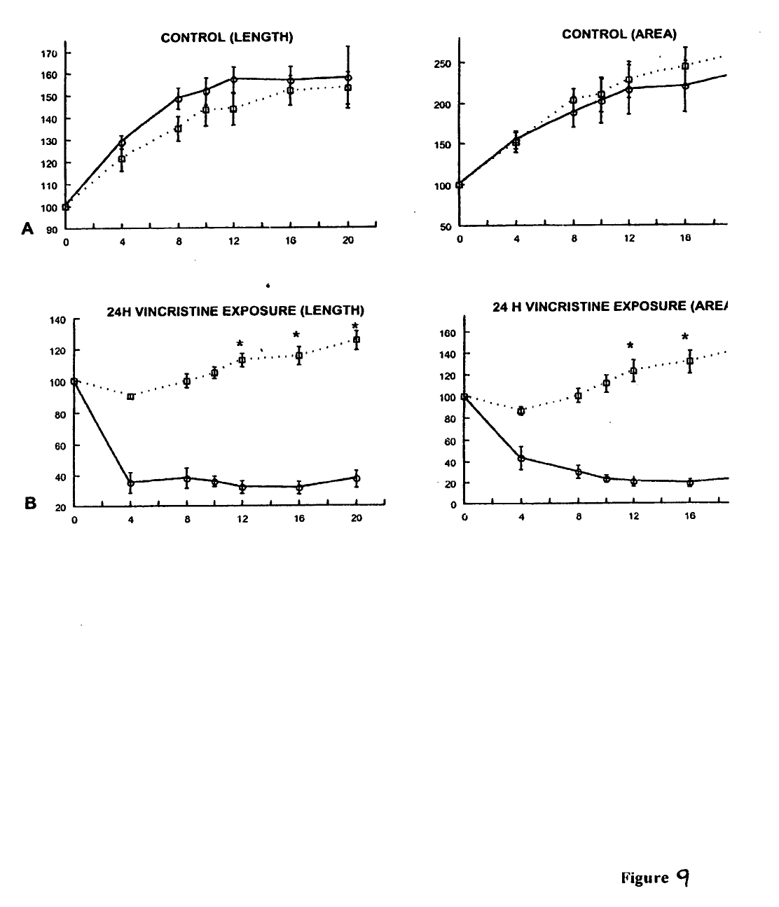

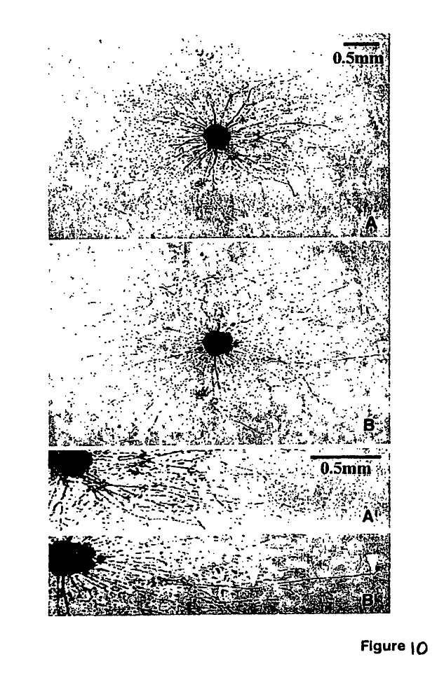

DRG cultures from Wld^{S} and C57BL/6 mice were compared for their ability to resist axonal degeneration following transient vincristine exposure. Cultures not exposed to vincristine continued to grow throughout the experimental time period with no differences noted between C57BL/6 and Wld^{S}(FIG. 9A). After 24 hours of exposure the majority of axons from C57BL/6 mice did not survive. By 4 days after exposure axon length was reduced to 35% and DRG area was reduced to 32% of control values (FIG. 9B). Over the 20-day observation period no renewed growth was noted, and there was further loss of DRG area. In comparison, DRG from Wld^{S} mice showed growth arrest for the first 10 days, and then resumed growth to reach 125% and 150% of control values for length and area, respectively (FIG. 9B; FIG. 10). Vincristine-exposed Wld^{S} DRG never caught up to control cultures in terms of length or area over the 20-day observation period, however once growth resumed after 10 days, the rate of growth was similar to that of controls.

### 1.3.2. Expression of the Wld^{S} Transgene in Rat DRG

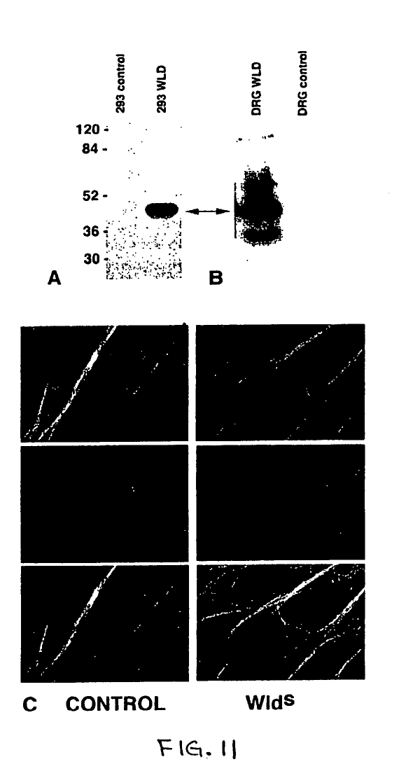

The Wld^{S} gene was successfully inserted into the adenovirus vector, as demonstrated by expression of a 42 kD protein in the replication-permissive HEK293A cells (FIG. 11A). Infection of DRG cultures with recombinant adenovirus similarly resulted in expression of the Wld^{S} protein as demonstrated by western blot (FIG. 11B) and by immunocytochemistry (FIG. 11C). In cultures infected with the recombinant virus, the Wld^{S} protein could be shown to colocalize with the axonal marker MAP-5 (FIG. 11C). At the dose used (109 virions/cc), there was some toxicity to the cultures when measured at 8 and 10 days after infection (Tables 6A and B). There was no difference, however, in the toxic effects of the two viral constructs.

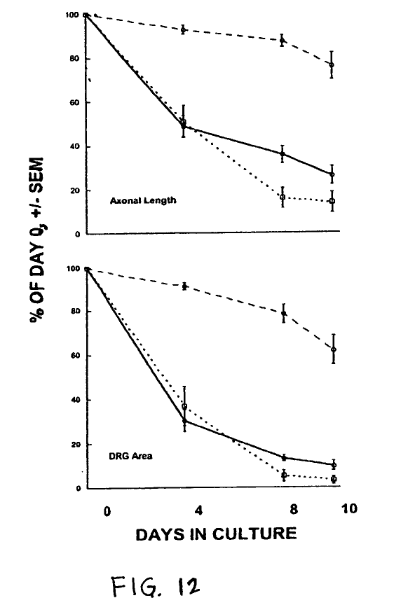

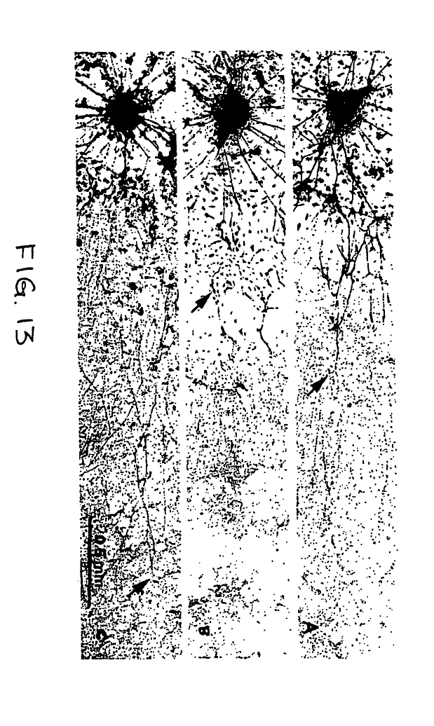

After exposure to vincristine, there was progressive axonal degeneration in uninfected cultures and in cultures infected with the control adenovirus, characterized by reduction in axonal lengths and DRG areas (Tables 7A and 7B and FIG. 12). No differences were noted between these two control groups. In the cultures infected with the adenovirus expressing the Wld^{S} gene, the degree of axonal degeneration was significantly less throughout the observation period (FIG. 12), reaching statistical significance by day 4 of exposure. FIG. 13 depicts the relative resistance to axonal degeneration of cultures expressing the Wld^{S} gene.

Discussion:

Using a clinically relevant model of toxic neuropathy in cultured DRG neurites we have further characterized the properties of the Wld^{S} gene mutation, and have demonstrated its therapeutic potential for preventing axonal degeneration. The experimental studies in mouse DRG provide conclusive evidence that the phenotype of the Wld^{S} is not merely a delay in axonal degeneration, but is also resistance against axonal death. This potential for neuroprotection could not be assessed in studies using axotomy-induced Wallerian degeneration as a model, since “regrowth” of axons isolated from their cell bodies is not only unlikely, but might be considered functionally irrelevant. The model of vincristine neuropathy provides a paradigm for a slowly progressive disorder that does not physically separate axons from their cell bodies, and allows for the consideration of long-term survival and regrowth. It remains unknown whether the Wld^{S} exerts its neuroprotective effect in this model at the level of the cell body or the axon itself, and this question is currently under investigation.

The introduction of the Wld^{S} gene into cultured rat DRG neurons also introduced into these previously susceptible neurons the phenotype of resistance to axonal degeneration. This demonstration conclusively identifies the Ufd2/D4 Colele chimeric gene, and its 42 kD protein product as the gene responsible for the Wld^{S} phenotype. It is not known how the Wld^{S} mutation provides for delayed Wallerian degeneration or neuroprotection in this model of toxic neuropathy. It is interesting to note, however, that Ufd (ubiquitin fusion degradation) proteins are involved in the ubiquitin degradation pathway that has been shown to be important in several models of cell death and, specifically, neurodegeneration.

Perhaps the most exciting conclusion from these studies is the “proof of principle” of gene transfer as a potential novel therapy for peripheral neuropathies. The introduction of the Wld^{S} gene using adenoviral technology clearly provided a beneficial response in vincristine-exposed DRG neurites. At the dose tested, however, there was a toxic effect of the virus itself. This problem has been noted by other investigators, and has limited the use of adenoviral-based gene therapy in humans. We are currently testing alternative doses of virus and viral constructs in order to limit the inherent toxicity of the virus.

The identification of a gene with the potential to provide protection against toxin induced peripheral neuropathy may have broad therapeutic implications. Cancer chemotherapeutic drugs, (including vincristine, paclitaxel, cisplatin, and suramin) are notorious for their toxic effects on the nervous system, with peripheral neuropathy being one of the most common side effects. Introduction of the Wld^{S} gene might provide for resistance to these toxic effects and allow for use of higher doses of these agents. In a broader sense, the Wld^{S} gene may provide a beneficial effect for patients with a variety of disorders of the PNS and CNS where axonal degeneration is a major component.

Tables 6A and 6B that follow depict values for DRG growth in normal (uninfected) and adenovirus-infected cultures not exposed to vincristine. Wld^{S} and lacZ are cultures exposed to adenoviruses expressing the Wld^{S} and lacZ genes, respectively. All adenoviruses expressed GFP. Numbers in parentheses are numbers of cultured DRG in each group. Values are % of day 0, +/−SEM.

Tables 7A and 7B that follow depict values for DRG axonal length and area in vincristine treated cultures. Row “a”, shows are uninfected cultures treated with vincristine. Rows “b” and “c” are infected with adenoviruses expressing the Wld^{S} and lacZ genes, respectively, and treated with vincristine. All adenoviruses expressed GFP. Numbers in parentheses are numbers of cultured DRG in each group. Values are % of day 0, +/−SEM.

TABLE 6A
Control values (length)

| | Days after treatment | | |
|---|---|---|---|
| | Day 4 | Day 8 | Day 10 |
| Group | Length of axons (% of day 0) | | |
| a: Normal (6) | 106.59 ± 3.19 | 121.99 ± 5.1 | 128.52 ± 2.88 |
| b: Wld^{S} (8) | 100.36 ± 1.96 | 97.08 ± 3.49 | 93.40 ± 4.35 |
| c: lacZ (7) | 98.40 ± 2.12 | 90.43 ± 4.69 | 83.72 ± 3.81 |
| p-value | NS | a:b < 0.01; | a:b < 0.001; |
| | | a:c < 0.01; b:c NS | a:c < 0.001; |
| | | | b:c NS |

TABLE 6B
Control values (area)

| | Days after treatment | | |
|---|---|---|---|
| | Day 4 | Day 8 | Day 10 |
| Group | Area of DRG halo (% of Day 0) | | |
| a: Normal (6) | 122.85 ± 3.47 | 161.24 ± 8.95 | 176.94 ± 6.43 |
| b: Wld^{S} (8) | 97.87 ± 1.47 | 90.14 ± 1.78 | 78.28 ± 3.25 |
| c: lacZ (7) | 92.90 ± 3.21 | 84.76 ± 7.48 | 73.03 ± 8.49 |
| p-value | a:b < 0.001; | a:b < 0.001; | a:b < 0.001; |
| | a:c < 0.001; | a:c < 0.001; | a:c < 0.001; |
| | b:c NS | b:c NS | b:c NS |

TABLE 7A
Vincristine treated (length)

| | Days after treatment | | |
|---|---|---|---|
| | Day 4 | Day 8 | Day 10 |
| Group | Length of axons (% of Day 0) | | |
| a: vin (6) | 48.79 ± 4.72 | 35.86 ± 3.76 | 26.28 ± 4.08 |
| b: Wld + vin (12) | 92.71 ± 2.1 | 87.23 ± 2.65 | 75.94 ± 6.11 |
| c: GFP + vin (12) | 51.05 ± 7.33 | 16.11 ± 4.75 | 13.96 ± 4.67 |
| p-value | a:b < 0.001; | a:b < 0.001; | a:b < 0.001; |
| | a:c NS; b:c < 0.001 | a:c < 0.05; | b:c < 0.001; |
| | | b:c < 0.001 | a:c NS |

TABLE 7B
Vincristine treated (area)

| | Days after treatment | | |
|---|---|---|---|
| | Day 4 | Day 8 | Day 10 |
| Group | Area of DRG halo (% of Day 0) | | |
| a: vin (6) | 30.43 ± 5.05 | 13.23 ± 1.52 | 9.78 ± 2.22 |
| b: Wld + vin (12) | 91.29 ± 1.78 | 78.32 ± 4.28 | 61.76 ± 6.58 |
| c: lacZ + vin (12) | 36.83 ± 8.70 | 5.21 ± 2.42 | 3.26 ± 1.66 |
| p-value | a:b < 0.001; | a:b < 0.001; | a:b < 0.001; |
| | a:c NS; | a:c NS; | a:c NS; b:c < 0.001 |
| | b:c < 0.001 | b:c < 0.001 | |

It should be emphasized that the above-described embodiments of the present invention are merely possible examples of implementations, and are merely set forth for a clear understanding of the principles of the invention. Many variations and modifications may be made to the above-described embodiment(s) of the invention without departing substantially from the spirit and principles of the invention. All such modifications and variations are intended to be included herein within the scope of this disclosure and the present invention and protected by the following claims.

Therefore, having thus described the invention, at least the following is claimed:

1. A method of protecting axons from axon degeneration by exposing the axons to a composition comprising a polynucleotide selected from: a polynucleotide sequence set forth in SEQ ID NO:1, or a degenerate variant of the SEQ ID NO:1; a polynucleotide sequence at least 90% identical to the polynucleotide sequence set forth in SEQ ID NO:1; a polynucleotide sequence at least 75% identical to the polynucleotide sequence set forth in SEQ ID NO:1; and a polynucleotide sequence at least 50% identical to the polynucleotide sequence set forth in SEQ ID NO:1.

2. A method of protecting axons from axon degeneration by exposing the axons to a composition comprising a polypeptide selected from: an amino acid sequence set forth in SEQ ID NO:2, or conservatively modified variants thereof; an amino acid sequence that is at least 90% identical to SEQ ID NO:2; an amino acid sequence that is at least 75% identical to SEQ ID NO:2; and an amino acid sequence that is at least 50% identical to SEQ ID NO:2.

3. A method of preventing axonal degeneration in a host having a nervous system dysfunction comprising administering to the host a therapeutically effective amount of a composition comprising a polynucleotide selected from: a polynucleotide sequence set forth in SEQ ID NO:1, or a degenerate variant of the SEQ ID NO:1; a polynucleotide sequence at least 90% identical to the polynucleotide sequence set forth in SEQ ID NO:1; a polynucleotide sequence at least 75% identical to the polynucleotide sequence set forth in SEQ ID NO:1; and a polynucleotide sequence at least 50% identical to the polynucleotide sequence set forth in SEQ ID NO:1.

4. A method of preventing axonal degeneration in a host having a nervous system dysfunction comprising administering to the host a therapeutically effective amount of a composition comprising a polypeptide selected from: an amino acid sequence set forth in SEQ ID NO:2, or conservatively modified variants thereof; an amino acid sequence that is at least 90% identical to SEQ ID NO:2; an amino acid sequence that is at least 75% identical to SEQ ID NO:2; and an amino acid sequence that is at least 50% identical to SEQ ID NO:2.

5. A method of treating a condition comprising administering to a host in need of treatment an effective amount of a polypeptide selected from: an amino acid sequence set forth in SEQ ID NO:2, or conservatively modified variants thereof; an amino acid sequence that is at least 90% identical to SEQ ID NO:2; an amino acid sequence that is at least 75% identical to SEQ ID NO:2; and an amino acid sequence that is at least 50% identical to SEQ ID NO:2.

6. The method of claim 5, wherein the condition is axonal degeneration.

7. The method of claim 5, wherein the condition is a nervous system disjunction.

8. A pharmaceutical composition comprising a polypeptide in combination with a pharmaceutically acceptable carrier, wherein the polypeptide is selected from: an amino acid sequence set forth in SEQ ID NO:2, or conservatively modified variants thereof; an amino acid sequence that is at least 90% identical to SEQ ID NO:2; an amino acid sequence that is at least 75% identical to SEQ ID NO:2; and an amino acid sequence that is at least 50% identical to SEQ ID NO:2.

9. A method of treating a condition comprising administering to a host in need of treatment an effective amount of a polynucleotide selected from: a polynucleotide sequence set forth in SEQ ID NO:1, or a degenerate variant of the SEQ ID NO:1; a polynucleotide sequence at least 90% identical to the polynucleotide sequence set forth in SEQ ID NO:1; a polynucleotide sequence at least 75% identical to the polynucleotide sequence set forth in SEQ ID NO:1; and a polynucleotide sequence at least 50% identical to the polynucleotide sequence set forth in SEQ ID NO:1.

10. The method of claim 9, wherein the condition is axonal degeneration.

11. The method of claim 9, wherein the condition is a nervous system disorder.

12. A pharmaceutical composition comprising a polynucleotide in combination with a pharmaceutically acceptable carrier, wherein the polynucleotide is selected from: a polynucleotide sequence set forth in SEQ ID NO:1, or a degenerate variant of the SEQ ID NO:1; a polynucleotide sequence at least 90% identical to the polynucleotide sequence set forth in SEQ ID NO:1; a polynucleotide sequence at least 75% identical to the polynucleotide sequence set forth in SEQ ID NO:1; and a polynucleotide sequence at least 50% identical to the polynucleotide sequence set forth in SEQ ID NO:1.

13. An antibody that selectively binds to a polypeptide selected from: an amino acid sequence set forth in SEQ ID NO:2, or conservatively modified variants thereof; an amino acid sequence that is at least 90% identical to SEQ ID NO:2; an amino acid sequence that is at least 75% identical to SEQ ID NO:2; and an amino acid sequence that is at least 50% identical to SEQ ID NO:2.

14. A fusion polypeptide comprising a heterologous polypeptide and a polypeptide selected from: an amino acid sequence set forth in SEQ ID NO:2, or conservatively modified variants thereof; an amino acid sequence that is at least 90% identical to SEQ ID NO:2; an amino acid sequence that is at least 75% identical to SEQ ID NO:2; and an amino acid sequence that is at least 50% identical to SEQ ID NO:2.

15. A probe comprising a polynucleotide sequence selected from: the polynucleotide sequence set forth in SEQ ID NO:1, or a degenerate variant of the SEQ ID NO:1; a polynucleotide sequence at least 90% identical to the polynucleotide sequence set forth in SEQ ID NO:1; a polynucleotide sequence at least 75% identical to the polynucleotide sequence set forth in SEQ ID NO:1; and a polynucleotide sequence at least 50% identical to the polynucleotide sequence set forth in SEQ ID NO:1.

16. An expression vector of an polynucleotide comprising a polynucleotide sequence selected from: the polynucleotide sequence set forth in SEQ ID NO:1, or a degenerate variant of the SEQ ID NO:1; a polynucleotide sequence at least 90% identical to the polynucleotide sequence set forth in SEQ ID NO:1; a polynucleotide sequence at least 75% identical to the polynucleotide sequence set forth in SEQ ID NO:1; and a polynucleotide sequence at least 50% identical to the polynucleotide sequence set forth in SEQ ID NO:1.

17. The expression vector of claim 16, wherein the expression vector is an adenovirus.

18. An expression vector of an polynucleotide comprising a polypeptide sequence selected from: an amino acid sequence set forth in SEQ ID NO:2, or conservatively modified variants thereof; an amino acid sequence that is at least 90% identical to SEQ ID NO:2; an amino acid sequence that is at least 75% identical to SEQ ID NO:2; and an amino acid sequence that is at least 50% identical to SEQ ID NO:2.
